(19) United States
(12) Patent Application Publication
(10) Pub. No.: US 2007/0196886 A1
(43) Pub. Date: Aug. 23, 2007

(54) Target And Method For Inhibition Of Bacterial Rna Polymerase Minimized Derivatives Of Peptide Antibiotic Mccj25

(76) Inventors: Richard H. Ebright, North Brunswick, NJ (US); Jayanta Mukhopadhyay, Edison, NJ (US)

Correspondence Address: HOFFMANN & BARON, LLP, 6900 JERICHO TURNPIKE, SYOSSET, NY, 11791, US

(21) Appl. No.: 10/571,226

(22) PCT Filed: Sep. 2, 2004
(86) PCT No.: PCT/US04/28640

§ 371 Date: Feb. 15, 2007

(51) Int. Cl. C12Q 1/37; C12Q 1/04; C07K 14/195

(52) U.S. Cl. 435/23; 435/34; 530/328; 530/326; 530/327

(57) ABSTRACT

Disclosed are targets, methods, and reagents for specific binding and inhibition of RNAP from bacterial species. The invention has applications in analysis of RNAP structure and function, control of bacterial gene expression, control of bacterial growth, antibacterial chemistry, antibacterial therapy, and drug discovery.

### CROSS-REFERENCE TO RELATED APPLICATIONS

This application is a Continuation-In-Part of Application No. PCT/US03/27457 filed on Sep. 4, 2003, which claims priority to provisional applications: 60/407,684 filed Sep. 4, 2002; 60/474,607 filed on Jun. 2, 2003; and 60/474,608 filed on Jun. 2, 2003, the contents of which are incorporated herein by reference.

### GOVERNMENT SUPPORT

This invention was supported with U.S. Government funds (NIH RO1-GM41376). Therefore, the Government may have rights in the invention.

### BACKGROUND ART

Bacterial infections remain among the most common and deadly causes of human disease. Infectious diseases are the third leading cause of death in the United States and the leading cause of death worldwide (Binder et al., Science 284:1311-1313 (1999)). Multi-drug-resistant bacteria now cause infection that poses a grave and growing threat to public health. It has been shown that bacterial pathogens can acquire resistance to first-line and even second-line antibiotics. (See, Stuart B. Levy, The Challenge of Antibiotic Resistance, in Scientific American, 46-53 (March, 1998); Walsh, C. (2000) Nature 406, 775-781; Schluger, N. (2000) Int. J. Tuberculosis Lung Disease 4, S71-S75; Raviglione et al., (2001) Ann NY Acad Sci 953, 88-97). New approaches to drug development are necessary to combat the ever-increasing number of antibiotic-resistant pathogens.

RNA is synthesized in cellular organisms by a complex molecular machine, known as RNA polymerase (“RNAP”). In its simplest bacterial form, RNAP comprises at least 4 subunits with a total molecular mass of around 400 kDa. RNAP mediates the transcription of DNA to produce RNA. Bacterial RNAP is a multimeric protein consisting of subunits α₂, β, β′, and ω. An σ factor is required for initiation of transcription by forming a holoenzyme complex.

Currently, there are a few known antibiotics that target RNAP—notably, rifampicin and rifampicin analogs (See Mitchison, D. (2000) Int. J. Tuberculosis Lung Disease 4, 796-806). Rifampicin is the only anti-tuberculosis compound able to rapidly clear infection and prevent relapse. Without rifampicin, treatment lengths must increase from 6 months to at least 18 months to ensure prevention of relapse. Rifampicin acts by specifically inhibiting RNAP (Campbell et al., (2001) Cell 104, 901-912). Rifampicin binds to a site adjacent to the active center of bacterial RNAP, the exit channel, and physically prevents synthesis of products longer than ˜4 nucleotides. Unfortunately, tuberculosis strains resistant to rifampicin (and rifampicin analogs) are becoming widespread, effectively removing rifampicin from the therapeutic arsenal. Thus, there is a need for novel antibiotics that target the same bacterial enzyme as rifampicin, RNAP, (and thus that have the same biochemical and therapeutic effects as rifampicin). There is also a need to develop methods for identifying antibiotics that interfere with bacterial RNAP.

Recently crystallographic structures have been determined for bacterial RNAP and eukaryotic RNAP II, and, based on the crystallographic structures, biophysical results, and biochemical results, models have been proposed for the structures of transcription initiation and elongation complexes (Gnatt et al.,(2001) Science 292, 1876-1882; Ebright, R. (2000) J. Mol. Biol. 304, 687-689; Naryshkin et al., (2000 Cell 101, 601-611; Kim et al., (2000) Science 288, 1418-1421; Korzheva et al., (2000) Science 289, 619-625; and Mekler et al., (2002) Cell 108:599-614). The models propose that nucleic acids completely fill the active-center cleft of RNAP, such that the only route by which incoming nucleoside triphosphate substrates (NTPs) can access the active center is through an approximately 25 Å long, 10 Å wide tunnel known as the “secondary channel” or “pore,” that bores through the floor of the active-center cleft of RNAP opposite the active-center cleft. (Gnatt et al., (2001 Science 292, 1876-1882; Ebright, R. (2000) J. Mol. Biol. 304, 687-689).

### SUMMARY OF THE INVENTION

Applicant has discovered that a region within the secondary channel comprising two adjacent short peptide segments of the RNAP β′ subunit are conserved in amino-acid sequence in bacterial species, including both Gram-positive bacteria and Gram-negative bacteria. Throughout the following specification, this region is referred to as the “β′ pocket” or the “target,” and the two short peptide segments are referred to as “homologous secondary channel amino acids or amino acid sequences.” Applicant has also discovered that this same region is not conserved, and in fact is radically different, in amino-acid sequence in eukaryotic RNAP, such as human RNAP I, RNAP II, and RNAP III.

Accordingly, a first aspect of the present invention is directed to a method for identifying agents that bind to a homologous secondary channel amino acid sequence, comprising preparing a reaction solution comprising the agent to be tested and an entity containing a homologous secondary channel amino acid sequence; and detecting presence or amount of binding. In a preferred embodiment, detection or quantitation of binding is conducted relative to binding of the agent to an entity containing an altered homologous amino acid sequence. In other preferred embodiments, quantitation of binding is compared to binding of the 21-amino acid bacteriocidal peptide microcin J25 (“MccJ25”), or a derivative thereof, to the entity via the homologous secondary channel amino acid sequence.

Another aspect of the present invention is directed to a method for identifying agents that inhibit an activity of RNAP via binding to a homologous secondary channel amino acid sequence. This aspect entails preparing a reaction solution comprising the agent to be tested, a catalytic entity containing a homologous secondary channel amino acid sequence, and a substrate for the entity; and determining extent of inhibition of RNAP activity via binding of the agent to the homologous secondary channel amino acid sequence.

In some preferred embodiments, quantitation of inhibition is compared to binding of MccJ25 to the catalytic entity via the homologous secondary channel amino acid sequence. MccJ25 is active against Gram-negative bacteria (Delgado et al., (2001) J. Bacteriol. 183:4543-4550). It was recently discovered that RNAP is a target of MccJ25. (Delgado et al., (2001) J. Bacteriol. 183:4543-4550; Yuzenkova et al., (2002) J. Biol. Chem. 277:50867-50875). The present invention provides that analogs of MccJ25 inhibit transcription by requiring determinants within the β′ pocket. The invention also provides probe-labeled derivatives of MccJ25, which can be used for detailed analysis of MccJ25 and other β′-pocket-directed inhibitors of RNAP. In one aspect of the invention, probe-labeled derivatives of MccJ25 can be used in high-throughput screening, by assessing ability of compounds or compound mixtures to compete with a probe-labeled derivative of MccJ25 for binding to RNAP or an RNAP fragment containing the target. Thus, the invention has broad applications in analysis of RNAP structure and function, control of bacterial gene expression, control of bacterial growth, antibacterial chemistry, antibacterial therapy, and drug discovery.

A further aspect of Applicants' invention is directed to an analog of MccJ25 having an amino acid sequence that differs from MccJ25 in terms of at least one amino acid deletion, insertion, or substitution, and that binds bacterial RNAP or inhibits bacterial RNAP catalytic activity to a greater extent than MccJ25.

The present invention also provides for the identification of potential antibacterial agents or antibiotics that, because of their binding affinity to regions within RNAP that are conserved among bacteria, have broad-spectrum activity. It also provides for the identification of potential anti-bacterial agents or antibiotics that, because of their substantial lack of binding affinity for eukaryotic RNAPs, will be relatively non-disruptive to normal cellular functions of the host.

It is anticipated that compounds identified according to the target and method of this invention would have applications not only in antibacterial therapy, but also in: (a) identification of bacterial RNAP (diagnostics, environmental-monitoring, and sensors applications), (b) labeling of bacterial RNAP (diagnostics, environmental-monitoring, imaging, and sensors applications), (c) immobilization of bacterial RNAP (diagnostics, environmental-monitoring, and sensors applications), (d) purification of bacterial RNA polymerase (biotechnology applications), (e) regulation of bacterial gene expression (biotechnology applications), and (f) antisepsis (antiseptics, disinfectants, and advanced-materials applications).

These and other aspects of the present invention will be better appreciated by reference to the following drawings and Detailed Description.

The file of this patent contains at least one drawing executed in color. Copies of this patent with color drawing(s) will be provided by the Patent and Trademark Office upon request and payment of the necessary fee.

### BRIEF DESCRIPTION OF THE DRAWINGS

FIG. 1 illustrates a sequence alignment for the target amino acid residues 736-747 and 779-781 of the β′ subunit of RNAP from Escherichia coli; and corresponding residues of the β′ subunits of Haemophilus influenzae, Vibrio cholerae, Pseudomonas aeruginose, Treponema pallidum, Borrelia burgdorferi, Xyella fastidiosa, Camploacter jejuni, Neisseria meningitides, Rickettsia prowazekii, Thermotoga maritime, Chlamydia trachomatis, Mycoplasma pneumoniae, Bacillus subtilis, Staphylococcus aureus, Mycobacterium tuberculosis, Synechocystis sp., Aquifex aeolicus, Deinococcus radiodurans, Thermus thermophilus, and Thermus aquaticus (collectively, “the homologous bacterial RNAP secondary channel amino acids”); and corresponding residues of the largest subunits of human RNAP I, RNAP II and RNAP III).

FIG. 2 illustrates a model showing the location of the target within the structure of RNAP.

FIG. 3 (A) illustrates results of electrophoretic mobility shift experiments assessing effects of MccJ25 on open-complex formation. RPₒ, RNAP-promoter open complex; P, promoter DNA. (B and C) illustrate that MccJ25 inhibits abortive initiation and elongation. (B) illustrates results of transcription experiments assessing effects of MccJ25 on abortive initiation and elongation. (C) illustrates results of transcription experiments assessing effects of MccJ25 on abortive initiation and elongation with RNAP derivative bearing Thr931→Ile substitution in RNAP β′ subunit (see Delgado et al., 2001). (D) illustrates results of transcription experiments assessing NTP-concentration-dependence of effects of MccJ25 on abortive initiation and elongation. (E) illustrates double-reciprocal plot for inhibition of synthesis of 3-mer and 4-mer abortive products. Filled circles, no MccJ25; open circles, 1 μM MccJ25; filled triangles, 10 μM MccJ25; open triangles, 100 μM MccJ25. Data are from (D). Lines are fits to a partial-competitive model of inhibition (Kᵢ=1.4±0.2 μM; α=15±3; r²=0.99). Within experimental error, Vₘₐₓ is independent of MccJ25 (2.2±0.1 mol/mol template/min at 0 μM MccJ25; 1.9±0.4 mol/mol template/min at 1 μM MccJ25; 1.4±0.7 mol/mol template/min at 10 μM MccJ25). (F) illustrates double-reciprocal plot for inhibition of synthesis of 3-mer and 4-mer abortive products. Filled circles, no MccJ25; open circles, 1 μM MccJ25; filled triangles, 10 μM MccJ25; open triangles, 100 μM MccJ25. Data are from fluorescence-detected abortive initiation assays (see Experimental Procedures). Lines are fits to a partial-competitive model of inhibition (Kᵢ=1.2±0.3 μM; α=87±2; r²=0.97). Within experimental error, Vₘₐₓ is independent of MccJ25 (4.0±1.3 mol/mol template/min at 0 μM MccJ25; 5.3±2.8 mol/mol template/min at 1 μM MccJ25; 2.4±2.9 mol/mol template/min at 10 μM MccJ25).

FIG. 4 illustrates results of transcription experiment assessing effects of MccJ25 on elongation [experiment analogous to experiments in FIG. 1B-D, but starting with a halted transcription elongation complex containing 12 nt of RNA (TEC12)]. TEC12 was prepared by reaction for 5 min at 37° C. with 0.5 mM ApA, and 12.5 μM each of [α-³²P]UTP, ATP, and GTP. Following equilibration for 2 min at 37° C. with 0, 1, 10, or 100 μM MccJ25, TEC12 was chased by reaction for 5 min at 37° C. with 25 μM or 100 μM each of UTP, ATP, GTP, and CTP.

FIG. 5 illustrates that MccJ25 binds within the RNAP secondary channel. Schematic maps of RNAP β′ and β subunits showing locations of conserved regions (conserved regions A-H of β′ and A-I of β lettered and shaded; additional conserved regions lightly shaded) and locations of single-residue substitutions that confer MccJ25-resistance (B)-(D) illustrates the three-dimensional structure of RNAP showing locations of single-residue substitutions that confer MccJ25-resistance [substitutions of Delgado et al., 2001 and Yuzenkova et al., 2002 in (B); substitutions from random mutagenesis in (C); and substitutions from random and saturation mutagenesis=in (D)]. Each panel presents a stereodiagram, with a view directly into the RNAP secondary channel, toward the active-center Mg⁺⁺ (white sphere at center). Atomic coordinates are based on the crystal structure of Thermus thermophilus RNAP at 2.6 Å resolution (Vassyleyev et al., 2002; PDB accession 1IW7; σ subunit omitted for clarity). Correspondences between residues of E. coli RNAP β′ and β and T. thermophilus RNAP β′ and β are based on comprehensive sequence alignments of bacterial, archaeal, and eukaryotic RNAP β′ and β homologs (unpublished).

FIG. 6 illustrates results of stopped-flow experiments assessing kinetics of interaction of Cy3-MccJ25 with RNAP. (A) illustrates reaction with 2.5 μM Cy3-MccJ25. Triangles, Cy3-MccJ25; circles, buffer control. (B) illustrates reaction with 5 μM Cy3-MccJ25. Triangles, Cy3-MccJ25; circles, buffer control.

FIG. 7 illustrates that MccJ25 binds within the RNAP secondary channel. (A) illustrates binding of Cy3-MccJ25 to DAC-σ⁷⁰ RNAP holoenzyme (filled circles) or DAC-σ⁷⁰ [Ile931]β′RNAP holoenzyme (open circles). (B) illustrates competition by MccJ25 for binding of Cy3-MccJ25 to DAC-σ⁷⁰ RNAP holoenzyme. (C) illustrates competition by GreB for binding of Cy3-MccJ25 to DAC-σ⁷⁰ RNAP holoenzyme. (D) illustrates results of systematic FRET measurements defining distances between Cy3 and DAC chromophores in complexes of Cy3-MccJ25 and DAC-labelled RNAP holoenzyme derivatives (ten with DAC at sites within σ⁷⁰, two with DAC at sites within ω). E, FRET efficiency; Rₒ, Förster parameter; R, distance. (E) illustrates results of systematic FRET measurements and distance-restrained docking defining possible positions of Cy3 chromophore in complexes of Cy3-MccJ25 and DAC-labelled RNAP holoenzyme derivatives (view as in FIG. 4). Dark spheres in grape-like clusters near center of each stereo panel, possible positions of Cy3 chromophore (125 highest-ranked solutions; see Supplementary Information: Distance-restrained docking); light spheres, DAC sites within σ⁷⁰; dark spheres, DAC sites within ω; dark van der Waals surfaces (as in FIG. 5D), sites of substitutions in β′ and β that confer MccJ25-resistance; white sphere, active-center Mg⁺⁺; ribbons, β′, β, α, ω, and σ⁷⁰.

FIG. 8 illustrates that MccJ25 obstructs the RNAP secondary channel. (A) illustrates the structure of MccJ25 (Bayro et al., (2003) J. Am. Chem. Soc. 125, 12382-12383; PDB accession 1PP5; see also Rosengren et al., (2003) J Am Chem Soc 125, 12464-12474; Wilson et al., (2003) J Am Chem Soc 125, 12475-12483). (B,C) illustrate the model for the structure of the MccJ25-RNAP complex (view orientation as in FIGS. 4,5). Light van der Waals surface, MccJ25 (oriented as in panel A); dark van der Waals surfaces, sites of substitutions in β′ and β that confer MccJ25-resistance; labels, sites of substitutions in β′ that confer highest-level MccJ25-resistance (MBC=1 mg/ml).

### BEST MODE OF CARRYING OUT THE INVENTION

The present invention provides methods of designing specific inhibitors of bacterial RNAP, the enzyme responsible for transcription. The present invention also provides for the design of small-molecule inhibitors (i.e., diameter<20 Å, MW≦2.1 kDa) that bind, with a k_{d}<1 μM, to a target site that is highly conserved in bacterial RNAP, but distinctly different in eukaryotic RNAP. The invention provides targets and methods for specific binding and inhibition of RNAP from bacterial species. The invention has applications in control of bacterial gene expression, control of bacterial growth, antibacterial chemistry, and antibacterial therapy.

Recently, crystallographic structures have been determined for bacterial RNAP and eukaryotic RNAP II (Zhang et al., (1999) Cell 98, 811-824; Cramer et al., (2000) Science 288, 640-649; Cramer. et al., (2001) Science 292, 1863-1876; Gnatt et al., (2001 Science 292, 1876-1882; and Ebright, R. (2000) J. Mol. Biol. 304, 687-689), and, based on the crystallographic structures, biophysical results, and biochemical results, models have been proposed for the structures of transcription initiation and elongation complexes (Gnatt et al., (2001)Science 292, 1876-1882; Ebright, R. (2000) J. Mol. Biol. 304, 687-689; Naryshkin et al., (2000 Cell 101, 601-611; Kim et al., (2000) Science 288, 1418-1421; Korzheva et al., (2000) Science 289, 619-625; and Mekler et al., (2002) Cell 108:599-614).

The models for the transcription elongation complex imply that nucleic acids completely fill the active-center cleft of RNAP, and thus the only route by which incoming nucleoside-triphosphate substrates (“NTPs”) can access the active center is through a ˜25 Å long, ˜10 Å wide tunnel—the “secondary channel” or “pore”—that bores through the floor of the active-center cleft of RNAP and extends to the exterior surface of RNAP opposite the active-center cleft (Gnatt et al., (2001)Science 292, 1876-1882; Ebright, R. (2000) J. Mol. Biol. 304, 687-689; and Korzheva et al., (2000) Science 289, 619-625).

The models for the transcription elongation complex imply that the RNAP secondary channel mediates multiple biochemical activities important for function of RNAP, including: uptake of NTPs, release of pyrophosphate product, release of abortive-RNA and edited-RNA products, interaction with RNA product during transcriptional pausing, interaction with RNA product during transcriptional arrest, interaction with RNA product during editing, and interaction with the elongation factors GreA and GreB.

It has now been found, and is disclosed herein, that physically blocking the RNAP secondary channel with a small molecule inhibits at least one of these activities. Specifically, it has now been found, and is disclosed herein, that physically blocking the RNAP secondary channel with a small molecule prevents uptake of NTPs by RNAP and thus inhibits transcription.

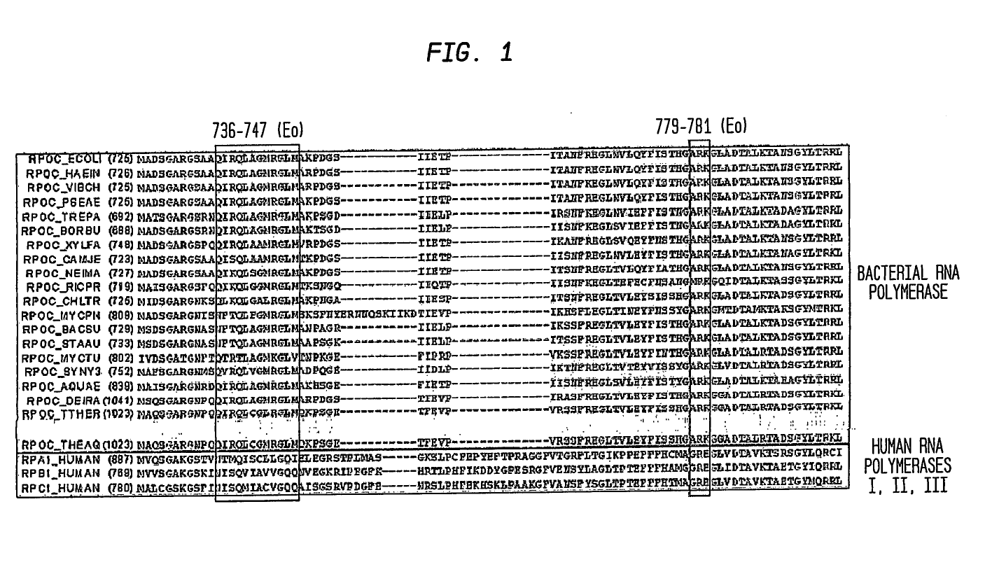

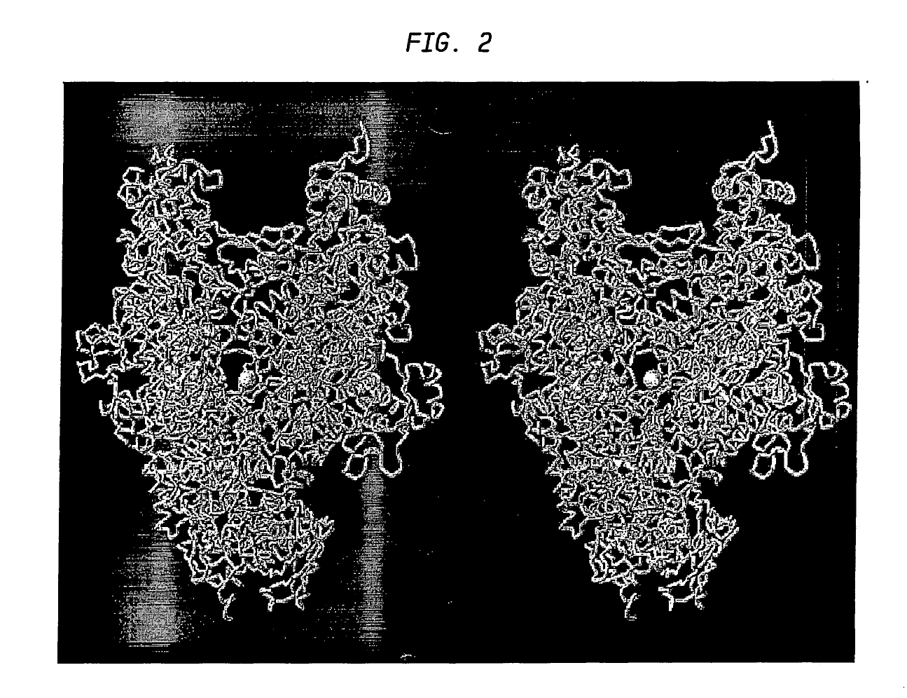

The present invention is based in part on Applicant's discovery that a region within the RNAP secondary channel comprising amino acids 736-747 and 779-781 of the RNAP β′ subunit from Escherichia coli (the “β′ pocket” or “target”) is a useful target for compounds that block transcription. It was found that said pocket is lined with residues that are invariant or nearly invariant in RNAP from bacterial species, but that are radically different in RNAP from eukaryotic species (FIG. 1). It further was found that this region forms a ˜5 Å shallow pocket within the wall of the RNAP secondary channel (FIG. 2).

Models for the transcription elongation complex suggest that nucleic acids completely fill the active channel of RNAP, and thus that the only route by which incoming nucleoside-triphosphate (NTP) substrates can access this active center is through this secondary channel. The present invention therefore relates to molecules that bind to the secondary channel of RNAP from Escherichia coli, or in corresponding regions of RNAP from other bacterial species. In a preferred embodiment, the present invention relates to molecules that bind to RNAP in the secondary channel of RNAP from Escherichia coli, or in corresponding regions of RNAP from other bacterial species and prevent RNAP from carrying out at least one biochemical activity with which the secondary channel is associated (e.g., uptake of NTP substrates, release of pyrophosphate product, release of edited nucleotide and oligonucleotide products, interaction with RNA product during transcriptional pausing, interaction with RNA product during transcriptional arrest, interaction with RNA product during editing, and interaction with the elongation factors GreA and GreB). In one aspect of the present invention, methods and compositions are described involving compounds that specifically block the secondary channel of RNAP resulting in inhibition of transcription.

The target referred to above in Escherichia coli is similar in amino acid sequence to that of most or all other species of bacterial RNAP and is called herein the homologous bacterial RNAP secondary channel (FIG. 1). (For example, amino acid residues 736-747 and 779-781 of the β′ subunit of RNAP from Escherichia coli exhibit high similarity to amino acid residues 740-751 and 783-785 of the β′ subunit of RNAP from Bacillus subtilis (FIG. 1).) Thus, the discovery of a molecule that binds to the target and inhibits an activity associated with the secondary channel in Escherichia coli RNAP also is likely to bind to the target an inhibit an activity associated with the secondary channel in other species of bacterial RNAP. Therefore, molecules found to be have antibiotic activity (through binding to the target and inhibiting an activity associated with the secondary channel) against Escherichia coli are likely to be found to have antibiotic activity against other bacterial species.

In contrast, the target differs radically in amino acid sequence between bacterial RNAP and eukaryotic RNAP, including human RNAP I, RNAP II, and RNAP III (FIG. 1). This allows for the identification of molecules that bind, in a target-dependent fashion, to bacterial RNAP, but that do not bind, or that bind substantially less well, to eukaryotic RNAP. This also allows for the identification of molecules that inhibit, in a target-dependent fashion, an activity of to bacterial RNAP, but that do not inhibit, or that inhibit substantially less well, an activity of eukaryotic RNAP. This differentiation is important, because it permits the identification of bacterial-RNAP-selective binding molecules and bacteria-selective inhibitors.

The invention provides, by way of example only, a target region corresponding to, and alignable with residues 736-747 and 779-781 of the β′ subunit of RNAP from Escherichia coli; as well as corresponding residues of the β′ subunit of Bacillus subtilis, Haemophilus influenzae, Vibrio cholerae, Pseudomonas aeruginosa, Treponema pallidum, Borrelia burgdorferi, Xyella fastidiosa, Campylobacter jejuni, Neisseria meningitidis, Rickettsia prowazekii, Thermotoga maritima, Chlamydia trachomatis, Mycoplasma pneumoniae, Staphylococcus aureus, Mycobacterium tuberculosis, Synechocystis sp., Aquifex aeolicus, Deinococcus radiodurans, Thermus thermophilus, and Thermus aquaticus.

The present invention further relates to a method for identifying molecules that bind to the β′ pocket through the use of an assay for molecules that bind to RNAP in a β′-pocket-specific fashion. In one embodiment, Escherichia coli RNAP or a fragment thereof containing the β′ pocket, is used as the test protein for binding, and a derivative of said RNAP or RNAP fragment having at least one a substitution, an insertion, or a deletion within the β′ pocket is used as the control protein for target-site specificity of binding. “Hits” can be analyzed for binding and inhibition of Gram-negative-bacterial RNAP, Gram-positive-bacterial RNAP, and eukaryotic RNAP I, RNAP III and RNAP III, in vivo and in vitro. “Hits” also can be characterized structurally by x-ray diffraction analysis of co-crystals with RNAP or an RNAP fragment containing the β′ pocket.

The invention also provides strategies to identify small-molecule inhibitors from compound libraries. By way of example, two strategies are described as follows: (a) selection of molecules that bind to RNAP, or a fragment thereof, in a β′-pocket-dependent fashion (affinity selection of phage-displayed linear and cyclic decapeptide libraries), and (b) screening for molecules that inhibit transcription in a β′-pocket-dependent fashion (iterative deconvolution of solution-phase linear and cyclic D-hexapeptide libraries). In each case, the invention provides the use of a wild-type bacterial RNAP, or fragment thereof, as the test protein for binding/inhibition, and a derivative of bacterial RNAP, or a fragment thereof, having at least one substitution, insertion, or deletion within the β′ pocket as the control protein for β′-pocket dependence of binding/inhibition.

The invention also provides for a method of identifying a compound for use as an inhibitor of bacterial RNAP comprising: analyzing a compound or a compound library, that involves docking to, modeling of, geometric calculations with, and/or energetic calculations with, a portion of the structure of an RNAP from a bacterial species comprising at least one residue within the set of residues corresponding to, and alignable with, the target.

The invention provides for at least three drug-discovery assay methods: a) screening based on binding of a compound within the secondary channel of a bacterial RNAP or fragment thereof; b) screening based on inhibition of an activity associated with the secondary channel of a bacterial RNAP or fragment thereof; and c) screening based on displacement of a compound, containing a detectable group, from the secondary channel of a bacterial RNAP or a fragment thereof.

One of the embodiments of the present invention is an assay system designed to identify compounds that bind a bacterial RNAP, or a fragment thereof, in a manner that requires the β′ pocket. The assay is designed to measure the binding of a compound to a determinant that includes at least one amino acid residue contained within a set of amino acid residues identifiable by sequence alignment and/or structure alignment as corresponding to amino acid residues 736-747 and 779-781 of Escherichia coli RNAP β′ or to amino acid residues 740-751 and 783-785 of Bacillius subtilis RNAP β′.

One of the embodiments of the present invention is an assay system designed to identify compounds that inhibit an activity of a bacterial RNAP, or a fragment thereof, in a manner that requires the β′ pocket. The assay is designed to measure the inhibition of an activity, said inhibition involving the binding of a compound to a determinant that includes at least one amino acid residue contained within a set of amino acid residues identifiable by sequence alignment and/or structure alignment as corresponding to amino acid residues 736-747 and 779-781 of Escherichia coli RNAP β′ or to amino acid residues 740-751 and 783-785 of Bacillius subtilis RNAP β′.

Another embodiment of the present invention is an assay designed to measure the binding of a compound to a bacterial RNAP derivative, or a fragment thereof, containing at least one amino acid substitution, insertion, or deletion within a set of amino acid residues identifiable by sequence alignment and/or structure alignment as corresponding to amino acid residues 736-747 and 779-781 of Escherichia coli RNAP β′ or to amino acid residues 740-751 and 783-785 of Bacillius subtilis RNAP β′.

Another embodiment of the present invention is an assay designed to measure the inhibition of an activity of a bacterial RNAP derivative, or a fragment thereof, containing at least one amino acid substitution, insertion, or deletion within a set of amino acid residues identifiable by sequence alignment and/or structure alignment as corresponding to amino acid residues 736-747 and 779-781 of Escherichia coli RNAP β′ or to amino acid residues 740-751 and 783-785 of Bacillius subtilis RNAP β′.

Isolation of RNAP:

The bacterial RNAP, or RNAP derivative, can be isolated from bacteria, produced by recombinant methods, or produced through in vitro protein synthesis. Various compounds can be introduced to determine whether a tested compound binds to, inhibits an activity of, or displaces a detectable-group containing molecule from, the bacterial RNAP or RNAP derivative in a β′-pocket-dependent manner.

Tested compounds can include antibodies, peptides, and various chemical compounds. Additionally, with the known amino acid sequence for a particular RNAP, one of skill in the art could design specific inhibitors. The tested compounds can be chosen from chemical libraries, or a computer model can be used to choose compounds that are likely to be effective based on the known structure of the secondary channel, the β′ pocket, and the structure of the compound.

The assay system can be performed in vivo or in vitro and thus does not necessarily require isolation of the RNAP.

The compounds can also be tested for competitive inhibition. Preferred strategies for identifying inhibitors include: 1) through affinity selection of phage-displayed linear and cyclic decapeptide libraries and 2) through iterative deconvolution of solution-phase linear and cyclic D-hexapeptide libraries. Wild type E. coli RNAP is the preferred test protein for binding and inhibition and [Val744; Gln746]β′-RNAP (a derivative of E. coli RNAP having substitutions at two positions in the β′ (738-747) pocket) as the control protein. Deconvolution essentially entails the resynthesis of that combinatorial pool or mixture that was found to be active in screening against a target of interest. Resynthesis may result in the generation of a set of smaller pools or mixtures, or a set of individual compounds. Rescreening and iterative deconvolution are performed until the individual compounds that are responsible for the activity observed in the screens of the parent mixtures are isolated.

Phage Display-General Method: Searching for Peptide Ligands with an Epitope Library:

Tens of millions of short peptides can be easily surveyed for tight binding to an antibody, receptor or other binding protein using an “epitope library.” (See (1990) Science 249:386; (1990) Science 249:404; and (1990) Proc. Natl. Acad. Sci. 87:6378). The library is a vast mixture of filamentous phage clones, each displaying one peptide sequence on the virion surface. The survey is accomplished by using the binding protein to affinity-purify phage that display tight-binding peptides and propagating the purified phage in Escherichia coli. The amino acid sequences of the peptides displayed on the phage are then determined by sequencing the corresponding coding region in the viral DNA's. Potential applications of the epitope library include investigation of the specificity of antibodies and discovery of mimetic drug candidates.

“Fusion phage” are filamentous bacteriophage vectors in which foreign antigenic determinants are cloned into phage gene III and displayed as part of the gene III protein (pIII) at one tip of the virion. Fusion phage whose displayed determinant binds an antibody (Ab) can be selected from a vast background of nonbinding phage by affinity purification (AP) as follows: First, phage are reacted with biotinylated Ab (bio-Ab), then diluted and placed on a streptavidin-coated petri dish, thereby specifically attaching Ab-reactive phage to the plastic surface through the Ab-biotin-streptavidin bridge. Free phage are washed away, and bound phage eluted in acid and used to infect Escherichia coli cells. A single round of AP can enrich Ab-binding phage by as much as a factor of 10⁵ relative to unreactive phage; further enrichment is achieved by further rounds of AP after amplification on agar medium. Thus, Ab serves as a powerful selective agent favoring the target clones, so that vast numbers of phage can be surveyed.

The idea of using fusion phage to develop an “epitope library” (Parmley and G. P. Smith, (1988) Gene 73:305) was inspired by the synthetic “mimotope” strategy of Geysen et al. (See Synthetic Peptides as Antigens; Ciba Foundation Symposium 119, R. Porter and J. Wheelan, Eds. (Wiley, New York. 1986), pp. 131-149). By synthesizing peptide mixtures on plastic pins and assessing their ability to bind an Ab against a protein antigen, these workers delineated a peptide that mimics a discontinuous epitope—an Ab-binding determinant composed of residues distant in the primary sequence but adjacent in the folded structure. They called these peptide mimics mimotopes. In this way ligands can be discovered for an Ab whose specificity is not known in advance.

Fusion phage displaying short cloned peptides are infectious analogs of chemically synthesized mimotopes, with the key advantages of replicability and clonability. A large library of such phage—an “epitope library”—may display tens of millions of peptide epitopes. The peptides can in effect be individually surveyed for binding to an Ab or other binding protein by affinity purifying reactive phage from the library, propagating individual phage clones, and sequencing the relevant part of their DNA's to determine the amino acid sequences of their displayed peptides. A survey based on the epitope library undoubtedly would be imperfect because of bias introduced by the biology of the phage and other factors; still, it would represent a powerful new approach to the study of the specificity of Ab's and other binding proteins. (See Scott and Smith (1990) Science 249:386; Devlin et al., (1990) Science 249:404; Ciwirla et al., (1990) Proc. Natl. Acad. Sci. 87:6378; McLafferty et al., (1993) Gene 128:29; Alessandra et al., (1993) Gene 128:51; McConnell et al., (1994) Gene 151:115, which are incorporated herein by reference).

Iterative Deconvolution Approach:

See the following reference for a general discussion of iterative deconvolution: (Ostresh et al., (1996) Meths. Enzym. 267:220, which is incorporated herein by reference). The practical development of synthetic combinatorial libraries (SCLs) made up of tens of millions of compounds has proven to be a powerful source for the identification of novel biologically active compounds such as analgesics, antibacterials, antifungals, and enzyme inhibitors. (See Pinilla et al., (1994) Drug Dev. Res. 33:133; Pinilla et al., (1995) Pept. Sci. 37:221; Gallop et al., (1994) J. Med. Chem. 37:1233). In particular, a range of new compounds having potent antimicrobial and/or antifungal activities have been rapidly identified from pools of millions of compounds. (See Blondelle et al., (1995) J. Appl. Bacteriol. 78:39; Blondelle et al., (1994) Antimicrob. Agent Chemother. 38:2280; Ostresh et al., (1994) Proc. Nail. Acad. Sci. U.S.A. 91:11138; Houghten et al., (1992) Bio Techniques 13:412; Houghten et al., (1991) Nature 354: 84).

Nonsupport-bound SCLs, originally composed of millions of peptides, were shown to be usable in virtually any assay system (including those involving membrane-bound acceptors or whole cell organisms). In an expansion of SCL concepts and diversities, the original peptide SCLs have been transformed (i.e., peralkylated and/or exhaustively reduced) using a “libraries from libraries” approach (Ostresh et al., (1994) Proc. Nail. Acad. Sci. U.S.A. 91:11138; Dörner et al., in “Peptides 1994: Proceedings of the 23rd European Peptide Symposium” (H. L. S. Maia, ed.), p. 463. Escom, Leiden, 1995; and Cuervo et al., Id. at page 465) to yield peptidomimetic and organic libraries having entirely different physical, chemical, and biological properties relative to the peptide SCLs used as starting materials. The screening of such libraries has yielded active compounds derived from entirely different sequences than the active peptides previously identified from the starting SCLs using the same assay.

Two approaches can be employed for the structural deconvolution of active compounds from assay data using nonsupport-bound SCLs: the “iterative” approach and the “positional scanning” approach. In addition, two synthetic methods were developed for the incorporation of multiple functionalities at diverse positions within an SCL. As first illustrated for peptides, (See Houghten et al., (1992) Bio Techniques 13:412; and Houghten et al., (1991) Nature 354: 84, which are incorporated herein by reference). The first synthetic method, known as the “divide, couple, and recombine” (DCR) (Id.) or “split resin” (Lam et al., (1991) Nature 354:82) method, has typically been used with the iterative deconvolution approach. The second synthetic method, which involves the use of a predefined chemical ratio of protected amino acids at each coupling step for incorporation of mixture positions, Ostresh et al., (1994) Biopolymers 34:1681) has been developed for use with the positional scanning deconvolution process (Pinilla et al., (1992) BioTechniques 13:901). This latter method offers the advantage that both defined and mixture positions are easily incorporated at any position in a sequence.

These synthesis and deconvolution methods have been used to identify individual active compounds in a wide variety of SCLs and assays. (Pinilla et al., (1994) Drug Dev. Res. 33:133; Pinilla et al., (1995) Pept. Sci. 37:221). More specifically, individual compounds from nonsupport-bound SCLs have been identified which have potent antimicrobial activity against gram-positive bacteria (Staphylococcus aureus, Streptococcus sanguis), gram-negative bacteria (Escherichia coli, Pseudomonas aeruginosa), and fungi (Candida albicans). The iterative deconvolution approach will be illustrated here for the preparation of a dual-defined position hexapeptide SCL, designated OOXXXX—NH₂ (where O represents a defined amino acid, and X represents a mixture of amino acids) using the DCR method. The mixtures making up this library have been assayed for antimicrobial and/or antifungal activity (Blondelle et al., (1995) Trends Anal. Chem. 14:83; Houghten et al., (1992) Bio Techniques 13:412; and Houghten et al., (1991) Nature 354:84) in order to identify the first two amino acid residues of active hexapeptide sequences. The remaining four positions were then identified sequentially through an iterative process of synthesis and screening. This process can be completed in 6 to 10 weeks (four separate iterative synthesis steps are required). The positional scanning approach, involves the screening of separate single position SCLs to identify the most effective amino acids at each position of the sequence. When used in concert, this information can be used to identify individual active sequences. This process can be completed in approximately 2 weeks (only one synthesis step is required for confirmation of activity).

Both iterative and positional scanning peptide SCLs have been used as starting materials for the generation of peptidomimetic SCLs using the “libraries from libraries” approach.

Synthesis:

Iterative Peptide Libraries: as Mentioned Earlier, these libraries are prepared using the DCR process (Houghten et al., (1991) Nature 354:84)(in conjunction with simultaneous multiple peptide synthesis (SMPS), (Houghten, (1985) Proc. Natl. Acad. Sci. U.S.A. 82:5131) also known as the “tea bag” approach. Standard t-butyloxycarbonyl (Boc)-based peptide synthesis protocols are typically used to couple protected amino acids (Bachem, Torrance, Calif.) to methylbenzhydrylamine (MBHA)-derivatized polystyrene resin (Peninsula, Belmont, Calif.). Fluorenylmethyloxycarbonyl (Fmoc)-based chemistry strategies can also be used. During preparation of the initial library, a portion of each resin mixture (i.e., X-resin, XX-resin, XXX-resin, etc) is held back for synthesis of the subsequent peptide mixtures during the iterative process in which additional positions are sequentially defined. While up to 76 amino acids have been used in the mixture positions, cysteine is normally omitted from the mixture positions of an SCL to prevent polymerization side reactions. It should be noted that for libraries synthesized by the DCR method, the number of resin beads used should be 10 to 100 times higher than the final number of individual compounds in a resin mixture in order to ensure statistical representation of each peptide in the library (Gallop et al., (1994) I. Med. Chem. 37:1233). The generation of a dual-defined position SCL made up of L-amino acid hexapeptides (designated OOXXXX—NH₂) is described here to illustrate the DCR methodology. This library contains approximately 52 million (20²×19⁴) different peptides.

The practical use of nonsupport-bound combinatorial libraries represents an important breakthrough in all areas of basic research and drug discovery. The use of a wide variety of chemical transformations permits a range of peptidomimetic libraries to be generated, which greatly expands the chemical diversity available. The results described in this chapter demonstrate that an existing peptide PS-SCL can be chemically transformed to generate a peptidomimetic SCL from which highly active individual compounds can be identified. The synthesis and deconvolution methods developed for peptide libraries are easily applied to other types of chemical pharmacophores. The soluble nature of the nonsupport-bound combinatorial libraries is a distinct advantage over other methods in that membrane-bound and whole cell assays can also be used. In addition, the deconvolution methods used allow the chemical structure of peptidic, peptidomimetic, and organic compounds to be determined based solely on the structural similarities of compounds within each active pool or sublibrary.

Screening for an Inhibitor of Bacterial RNAP:

One aspect of the invention provides high throughput screening of molecules specific to the bacterial RNAP target. This can be done in many different ways well known in the art. For example, this could be done by attaching bacterial RNAP to the bottom of the wells of a 96-well plate at an appropriate concentration by incubating the RNAP in the well overnight at 4° C. Alternatively, the wells are first coated with compositions of polylysine that facilitates the binding of the bacterial RNAP to the wells. Following attachment, samples from a library of test compounds (concentrations are determined by the compound being tested) are added (along with an appropriate binding buffer known in the art) to the wells and incubated for a sufficient time and temperature to facilitate binding. Following the incubation, the wells are washed with an appropriate washing solution at 4° C. Increasing or decreasing salt and/or detergent concentrations in the wash varies the stringency of the washing steps. Detection of binding is accomplished using antibodies (representative examples include RIA and ELISA), biotinylation, biotin-streptavidin binding, and radioisotopes. The concentration of the sample library compounds is also varied to calculate a binding affinity by Scatchard analysis. Binding to the bacterial RNAP target identifies a “lead compound”. Once a lead compound is identified, the screening process is repeated using compounds chemically related to the lead compound to identify compounds with the tightest binding affinities. Selected compounds having binding affinity are further tested in one of two assays. These assays use test compounds from 1) phage-displayed linear and cyclic decapeptide libraries and 2) iterative deconvolution of solution-phase linear and cyclic D-hexapeptide libraries.

A phage library can be used to test compounds that could bind to the β′ pocket of bacterial RNAP. The phage library is constructed in the N-terminal region of the major coat protein pVIII, as previously described (Felici et al., 1991). In addition, in an attempt to define a more constrained context, two Cys are included as invariant residues flanking the random nonapeptide. Transformation yields approximately 1×10⁸ independent clones, and the presence of a productive insert is indicated by the blue color of the colonies on Xgal/IPTG plates (Felici et al., 1991). The construction of the library results in hybrid capsids, expressing the random peptides, dispersed along wt pVIII copies. The absence of the Cys in wt pVIII allows one to detect the presence of free thiol groups in the hybrid capsids. Clones are analyzed with a Cys-specific recompound (DIG protein detection kit, Boehringer Mannheim, Germany) in order to show some of the peptides are in cyclized form. This indicates that in many cases the insert is displayed as a loop structure, which limits its mobility. Phage affinity purification is performed utilizing the biopanning technique, as previously described by Parmley and Smith (1988). After the round of biopanning, 10⁴ phage out of the initial 10¹⁰ are eluted from a streptavidin-coated plate. The phage are screened directly with a plaque assay. Single plaques (10⁵) are transferred onto nitrocellulose and probed with RNAP. Positive plaques are eluted from nitrocellulose, the phage are amplified and sequenced, and their reactivity is further confirmed by dot-blot analysis. The amino acid sequences are then deduced.

Biologically active compounds are selected from large populations of randomly generated sequences. Libraries are made up of six-residue peptide sequences with amidated carboxy-termini and either acetylated or non-acetylated amino-termini. The first two amino acids in each peptide chain are individually and specifically defined, while the last four amino acids consist of equimolar, or close to equimolar, mixtures of 19 of the 20 naturally occurring L-amino acids, cysteine is omitted from the mixture positions of the two libraries but included in the defined positions. The peptides in these libraries are generally represented as Ac—O₁O₂XXXX—NH₂ and O₁O₂XXXX—NH₂, where O₁ and O₂ are defined positions, which are represented by the single letters AA, AC, AD and so on up to and including YV, YW, YY, to reach a total of 400 (20²) combinations, and each X position is represented by an equimolar mixture of the 19 natural amino acids (non-natural amino acids can be used as well). Four mixture positions (XXXX) result in a total of 130, 321 (19⁴) different combinations. Each of the 400 different peptide mixtures that make up each of the libraries thus consists of 130, 321 individual hexamers; in total, 52,321,400 peptides are represented. The peptides are attached to a resin or alternatively cleaved from the resin, extracted and lyophilized before use. Each nonsupport-bound peptide mixture is typically used at a concentration of 1 mg/ml. Therefore, if one assumes that the average molecular weight of Ac—O₁O₂XXXX—NH₂ is 785, then a mixture of 130, 321 peptides at a total final concentration of 1 mg/ml yields a concentration of every peptide within each mixture of 7.67 ng/ml (9.8 nM), sufficiently high for most biologically significant interactions if even a single peptide sequence is active. After the mixture of libraries is screened for binding to bacterial RNAP, the remaining mixture positions are defined through an iterative enhancement and selection process in order to identify the most active sequence. An extremely rapid alternative method for identifying active compounds is the positional scanning approach. In this approach, if one uses a library made up of peptides, for example, each of the individual sub-libraries (one for each position along the peptide) that make up the positional scanning library is composed of 18 different peptide mixtures. Each position is defined (represented as O) and occupied by one of 18 of the 20 natural L-amino acids (cysteine and tryptophan are omitted); the remaining five positions of the six-residue sequence are composed of mixtures (represented as X) of the same 18 amino acids. The six different sub-libraries vary only in the location of their defined amino acids, they can therefore be represented as: Ac—O₁XXXXX—NH₂, Ac—XO₂XXXX—NH₂, Ac—XXO₃XXX—NH₂, Ac—XXXO₄XX—NH₂, AC-XXXXO₅X—NH₂, and Ac—XXXXXO₆—NH₂. As each peptide mixture represents 1,889,568 (18⁵) individual sequences, each of the six positional sub-libraries contains in total 34,012,224 (18×1,889,568) different compounds. Alternatively, each of the six individual sub-libraries (for example, Ac—XXXO₄XX—NH₂) can be examined independently and moved forward in an interactive fashion. When used in concert, however, this set of 108 mixtures constitutes a positional scanning library.

Screening Assays for Compounds that Interfere with the Interaction of RNAP Target and MccJ25:

The β′ pocket of bacterial RNAP—which contains the target—and compounds which interact and bind are sometimes referred to herein as “binding partners.” Any of a number of assay systems may be utilized to test compounds for their ability to interfere with the interaction of the binding partners. However, rapid, high-throughput assays for screening large numbers of compounds, including but not limited to ligands (natural or synthetic), peptides, or small organic molecules are preferred. Compounds that are so identified to interfere with the interaction of the binding partners should be further evaluated for binding or inhibitory activity in cell based assays, animal model systems and in patients as described herein. The basic principle of the assay systems used to identify the compounds of the present invention is based on the identification of compounds that interfere with the interaction between the target and MccJ25, which involves preparing a reaction mixture containing a bacterial RNAP, or RNAP fragment or derivative containing the secondary channel, and MccJ25 under conditions and for a time sufficient to allow the two binding partners to interact and bind, thus forming a complex. In order to test a compound for inhibitory activity, the reaction is conducted in the presence and absence of the test compound; i.e., the test compound may be initially included in the reaction mixture, or added at a time subsequent to the addition of the bacterial RNAP, or RNAP fragment or derivative containing the secondary channel, and MccJ25; controls are incubated without the test compound. The formation of a complex between the bacterial RNAP secondary channel and MccJ25 then is detected. The formation of a complex in the control reaction, but not, or to a lesser extent, in the reaction mixture containing the test compound indicates that the compound interferes with the interaction between the bacterial RNAP, or RNAP fragment or derivative containing the secondary channel, and MccJ25.

The assay components and various formats that may be utilized are described in the subsections below.

Assay Components:

The bacterial RNAP, or RNAP fragment or derivative containing the secondary channel, and MccJ25 binding partners used as components in the assay may be derived from natural sources, e.g., purified from bacterial RNAP, respectively, using protein separation techniques well known in the art; produced by recombinant DNA technology using techniques known in the art (see e.g., Sambrook et al., 1989, Molecular Cloning: A Laboratory Manual, Cold Spring Harbor Laboratories Press, Cold Spring Harbor, N.Y.); and/or chemically synthesized in whole or in part using techniques known in the art; e.g., peptides can be synthesized by solid phase techniques, cleaved from the resin and purified by preparative high performance liquid chromatography (see, e.g., Creighton, 1983, Proteins: Structures and Molecular Principles, W. H. Freeman & Co., N.Y., pp. 50-60). The composition of the synthetic peptides may be confirmed by amino acid analysis or sequencing; e.g., using the Edman degradation procedure (see e.g., Creighton, 1983, supra at pp. 34-49).

One of the binding partners used in the assay system should be labeled, either directly or indirectly, to facilitate detection of a complex formed between the bacterial RNAP secondary channel and MccJ25. Any of a variety of suitable labeling systems may be used including but not limited to radioisotopes such as ¹²⁵I and ³²P; enzyme labeling systems that generate a detectable calorimetric signal or light when exposed to substrate; and fluorescent labels.

Fluorescent labels are preferred. Cyanine fluorescent labels are especially preferred. Cyanine labels incorporated at residue 13 or 15 of MccJ25 are especially preferred.

Where recombinant DNA technology is used to produce the bacterial RNAP, or RNAP fragment or derivative containing the secondary channel, and MccJ25 binding partners of the assay it may be advantageous to engineer fusion proteins that can facilitate labeling, immobilization and/or detection. For example, the coding sequence of the bacterial RNAP secondary channel can be fused to that of a heterologous protein that has enzyme activity or serves as an enzyme substrate in order to facilitate labeling and detection. The fusion constructs should be designed so that the heterologous component of the fusion product does not interfere with binding of the bacterial RNAP secondary channel and MccJ25.

Indirect labeling involves the use of a third protein, such as a labeled antibody, which specifically binds to the bacterial RNAP secondary channel. Such antibodies include but are not limited to polyclonal, monoclonal, chimeric, single chain, Fab fragments and fragments produced by an Fab expression library.

For the production of antibodies, various host animals may be immunized by injection with the bacterial RNAP secondary channel. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies may be prepared by using any technique, which provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72, Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce single chain antibodies specific to the bacterial RNAP secondary channel.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Assay Formats:

The assay can be conducted in a heterogeneous or homogeneous format. A heterogeneous assay is an assay in which reaction results are monitored by separating and detecting at least one component during or following reaction. A homogeneous reaction is an assay in which reaction results are monitored without separation of components. In either approach, the order of addition of reactants can be varied to obtain different information about the compounds being tested. For example, test compounds that interfere with the interaction between the binding partners, e.g., by competition, can be identified by conducting the reaction in the presence of the test substance; i.e., by adding the test substance to the reaction mixture prior to or simultaneously with the bacterial RNAP secondary channel and MccJ25. On the other hand, test compounds that disrupt preformed complexes, e.g. compounds with higher binding constants that displace one of the binding partners from the complex, can be tested, by adding the test compound to the reaction mixture after complexes have been formed. The various formats are described briefly below.

In one example of a heterogeneous assay system, one binding partner, e.g., either the bacterial RNAP secondary channel or MccJ25, is anchored onto a solid surface, and its binding partner, which is not anchored, is labeled, either directly or indirectly. In practice, microtiter plates are conveniently utilized. The anchored species may be immobilized by non-covalent or covalent attachments. Alternatively, an immobilized antibody specific for the bacterial RNAP secondary channel may be used to anchor the bacterial RNAP secondary channel to the solid surface. The surfaces may be prepared in advance and stored.

In order to conduct the assay, the binding partner of the immobilized species is added to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface; e.g., using a labeled antibody specific for the binding partner (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for an epitope on the bacterial RNAP secondary channel to anchor any complexes formed in solution. Again, depending upon the order of addition of reactants to the liquid phase, test compounds which inhibit complex or which disrupt preformed complexes can be identified.

In a preferred embodiment of the invention, a homogeneous assay can be used. In this approach, a preformed complex of the bacterial RNAP secondary channel and MccJ25 is prepared in which one of the binding partners is labeled, but the signal generated by the label is quenched due to complex formation (see, e.g., U.S. Pat. No. 4,109,496 by Rubenstein, which utilizes this approach for immunoassays). The addition of a test substance that competes with and displaces one of the binding partners from the preformed complex will result in the generation of a signal above background. In this way, test substances, which disrupt the bacterial RNAP secondary channel and MccJ25 interaction can be identified.

One aspect of the invention provides for developing methods to use fluorescence resonance energy transfer (FRET)-based homogeneous assays to provide probe-labelled derivatives of the MccJ25. (Förster, 1948; reviewed in Lilley and Wilson. 2000; Selvin, 2000; Mukhopadhyay et al., 2001; Mekler et al., 2002). FRET occurs in a system having a fluorescent probe serving as a donor and a second fluorescent probe serving as an acceptor, where the emission wavelength of the donor overlaps the excitation wavelength of the acceptor. In such a system, upon excitation of the donor with light of its excitation wavelength, energy can be transferred from the donor to the acceptor, resulting in excitation of the acceptor and omission at the acceptor's emission wavelength.

With commonly used fluorescent probes, FRET permits accurate determination of distances in the range of ˜20 to 100 Å. FRET permits accurate determination of distances up to more than one-half the diameter of a transcription complex (diameter ˜150 Å; Zhang et al. 1999; Cramer et al., 2001; Gnatt et al., 2001).

A preferred assay involves monitoring of FRET between a fluorescent-probe-labeled derivative of a bacterial RNAP and a fluorescent-probe-labeled derivative of MccJ25.

An especially preferred assay involves monitoring of FRET between a coumarin-dye-labeled derivative of a bacterial RNAP and a cyanine-dye-labeled derivative of MccJ25. Especially preferred sites of labeling of RNAP for this purpose include residue 14, residue 59, or residue 517 of Escherichia coli RNAP σ⁷⁰ subunit, or a corresponding residue, identifiable by sequence and/or structural alignment, of another bacterial RNAP σ subunit. Especially preferred sites of labeling of MccJ25 for this purpose include residue 13 and residue 15.

In accordance with the invention, a given compound found to inhibit one bacterium may be tested for general antibacterial activity against a wide range of different bacterial species. For example, and not by way of limitation, a compound that inhibits the interaction of Escherichi coli RNAP, or a RNAP fragment or derivative thereof containing the secondary channel, of with MccJ25 can be tested, according to the assays described infra, against Haemophilus influenzae.

Assays for Antibacterial Activity:

Any of the inhibitory compounds, which are identified in the foregoing assay systems, may be tested for antibacterial activity in any one of the various microbiological assays known to the skilled worker in the field of microbiology.

Animal Model Assays:

The most effective inhibitors of bacterial RNAP identified by the processes of the present invention can then be used for subsequent animal experiments. The ability of an inhibitor to prevent bacterial infection can be assayed in animal models that are natural hosts for bacteria. Such animals may include mammals such as pigs, dogs, ferrets, mice, monkeys, horses, and primates. As described in detail herein, such animal models can be used to determine the LD₅₀ and the LD₅₀ in animal subjects, and such data can be used to derive the therapeutic index for the inhibitor of the bacterial RNAP secondary channel/MccJ25 interaction.

Pharmaceutical Preparations and Methods of Administration:

The identified compounds that inhibit bacterial replication can be administered to a patient at therapeutically effective doses to treat bacterial infection. A therapeutically effective dose refers to that amount of the compound sufficient to result in amelioration of symptoms of bacterial infection.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of infection in order to minimize damage to uninfected cells and reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal infection, or a half-maximal inhibition) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

As used herein a “small molecule” is a compound that has a molecular weight of less than 15 kDa.

As used herein a “small organic molecule” is an organic compound [or organic compound complexed with an inorganic compound (e.g., metal)] that has a molecular weight of less than 3 kDa.

As used herein the term “about” means within 10 to 15%, preferably within 5 to 10%. For example an amino acid sequence that contains about 60 amino acid residues can contain between 51 to 69 amino acid residues, more preferably 57 to 63 amino acid residues.

As used herein the term “target” minimally comprises amino acid residues of a target set of residues corresponding to, and alignable with, either residues 736-747 and 779-781 of the β′ subunit of RNAP from Escherichia coli or a set of residues corresponding to, and alignable with residues 740-751 and 783-785 of the β′ subunit of RNAP from Bacillus subtilis.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc). [Reeck et al., Cell, 50:667 (1987)].

Accordingly, the term “sequence similarity” in all its grammatical forms refers to the degree of identity or correspondence between nucleic acid or amino acid sequences of proteins that do not share a common evolutionary origin [see Reeck et al., 1987, supra]. However, in common usage and in the instant application, the term “homologous,” when modified with an adverb such as “highly,” may refer to sequence similarity and not a common evolutionary origin.

Similarly, in a particular embodiment, two amino acid sequences are “substantially homologous” or “substantially similar” when greater than 25% of the amino acids are identical, or greater than about 50% are similar (functionally identical). Preferably, the similar or homologous sequences are identified by alignment using, for example, the GCG (Genetics Computer Group, Program Manual for the GCG Package, Version 7, Madison, Wis.) pileup program with the default parameters.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

The present invention contemplates isolation of nucleic acids encoding either targets I or I. The present invention further provides for subsequent modification of the nucleic acid to generate a fragment or modification of the target, that will crystallize.

Protein-Structure Based Design of Inhibitors of Bacterial RNAP:

Once the three-dimensional structure of a crystal comprising a bacterial RNAP target is determined, a potential modulator of the target, can be examined through the use of computer modeling using a docking program such as GRAM, DOCK, or AUTODOCK [Dunbrack et al., Folding & Design, 2:27-42 (1997)], to identify potential modulators of the bacterial RNAP target. This procedure can include computer fitting of potential modulators to the bacterial RNAP target to ascertain how well the shape and the chemical structure of the potential modulator will bind to either the individual bound subunits or to the bacterial RNAP target [Bugg et al., Scientific American, December:92-98 (1993); West et al., TIPS, 16:67-74 (1995)]. Computer programs can also be employed to estimate the attraction, repulsion, and steric hindrance of the subunits with a modulator/inhibitor (e.g., the bacterial RNAP target and a potential stabilizer).

Initially, compounds known to bind to the target—for example, MccJ25—can be systematically modified by computer modeling programs until one or more promising potential analogs are identified. In addition, systematic modification of selected analogs can then be systematically modified by computer modeling programs until one or more potential analogs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)]. Alternatively, a potential modulator could be obtained by initially screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990)]. A peptide selected in this manner would then be systematically modified by computer modeling programs as described above, and then treated analogously to a structural analog as described below.

Once a potential modulator/inhibitor is identified, it can be either selected from a library of chemicals as are commercially available from most large chemical companies including Merck, Glaxo Welcome, Bristol Meyers Squib, Monsanto/Searle, Eli Lilly, Novartis and Pharmacia UpJohn, or alternatively the potential modulator may be synthesized de novo. As mentioned above, the de novo synthesis of one, or even a group of, specific compounds is reasonable in the art of drug design. The potential modulator can be placed into a standard binding assay with RNAP or an active fragment thereof such as the target, for example. The subunit fragments can be synthesized by either standard peptide synthesis described above, or generated through recombinant DNA technology or classical proteolysis. Alternatively, the corresponding full-length proteins may be used in these assays.

For example, the β′ subunit can be attached to a solid support., Methods for placing the β′ subunit on the solid support are well known in the art and include such things as linking biotin to the β ′ subunit and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to the β′ subunit can be determined. Alternatively, or in addition, the dissociation constant between the labeled potential modulator and the β′ subunit, for example can be determined. Suitable labels for bacterial RNAP target or the potential modulator are exemplified herein. In a particular embodiment, isothermal calorimetry can be used to determine the stability of the bacterial RNAP target in the absence and presence of the potential modulator.

In another aspect of the present invention, a compound is assayed for its ability to bind to the target. A compound that binds to the target then can be selected. In a particular embodiment, the effect of a potential modulator on an activity of the bacterial RNAP target is determined. The potential modulator then can be added to a bacterial culture to ascertain its effect on bacterial proliferation. A potential modulator that inhibits bacterial proliferation then can be selected.

In a particular embodiment, the effect of the potential modulator on an activity of a bacterial RNAP, or a fragment thereof, is determined (either independently, or subsequent to a binding assay as exemplified above). In one such embodiment, the extent or rate of the DNA-dependent RNA transcription is determined. For such assays, a labeled nucleotide could be used. This assay can be performed using a real-time assay—e.g., with a fluorescent analog of a nucleotide. Alternatively, the determination can include the withdrawal of aliquots from the incubation mixture at defined intervals and subsequent placing of the aliquots on nitrocellulose paper or on gels. In a particular embodiment the potential modulator is selected when it is an inhibitor of the bacterial RNAP.

One assay for RNAP activity is a modification of the method of Burgess et al. [J. Biol. Chem., 244:6160 (1969)] [See http://www.worthington-biochem.com/manual/R/RNAP.html]. One unit incorporates one nanomole of UMP into acid insoluble products in 10 minutes at 37° C. under the assay conditions such as those listed below. The suggested recompounds are: (a) 0.04 M Tris-HCl, pH 7.9, containing 0.01 M MgCl.sub.2, 0.15 M KCl, and 0.5 mg/ml BSA; (b) Nucleoside triphosphates (NTP): 0.15 mM each of ATP, CTP, GTP, UTP; spiked with ³H-UTP 75000-150000 cpms/0.1 ml; (c) 0.15 mg/ml calf thymus DNA; (d) 10% cold perchloric acid; and (e) 1% cold perchloric acid. 0.1-0.5 units of RNAP in 5 μl-10 μl is used as the starting enzyme concentration.

The procedure is to add 0.1 ml Tris-HCl, 0.1 ml NTP and 0.1 ml DNA to a test tube for each sample or blank. At zero time enzyme (or buffer for blank) is added to each test tube, and the contents are then mixed and incubated at 37° C. for 10 minutes. 1 ml of 10% perchloric acid is added to the tubes to stop the reaction. The acid insoluble products can be collected by vacuum filtration through MILLIPORE filter discs having a pore size of 0.45 u-10 u (or equivalent). The filters are then washed four times with 1% cold perchloric acid using 1 ml-3 ml for each wash. These filters are then placed in scintillation vials. 2 mls of methyl cellosolve are added to the scintillation vials to dissolve the filters. When the filters are completely dissolved (after about five minutes) 10 mls of scintillation fluid are added and the vials are counted in a scintillation counter.

Additional assays for analysis of RNAP activity contemplated by the present invention include RNA transcription assays and fluorescence-detected abortive initiation assays described in detail below, concerning defining the target of MccJ25. The present invention further provides for assays for analysis of antibacterial activity, such as for example include a Minimal Bacteriocidal Concentration (MBC) assay also described in detail below, concerning defining the target of MccJ25.

For calculation of units of RNAP/mg of protein the equation described in U.S. Pat. No. 6,225,076 can be used.

When suitable potential modulators are identified, a crystal can be grown that comprises the bacterial RNAP, or a fragment thereof, and the potential modulator. Preferably, the crystal effectively diffracts X-rays for the determination of the atomic coordinates of the protein-ligand complex to a resolution of better than 4.0 Angstroms. The three-dimensional structure of the crystal is determined by molecular replacement. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR (see above), CNS, (Crystallography and NMR System, a next level of XPLOR), and AMORE [J. Navaza, Acta Crystallographics ASO, 157-163 (1994)]. Once the position and orientation are known an electron density map can be calculated using the search model to provide X-ray phases. Thereafter, the electron density is inspected for structural differences and the search model is modified to conform to the new structure. Using this approach, it will be possible to solve the three-dimensional structure of different bacterial target having pre-ascertained amino acid sequences. Other computer programs that can be used to solve the structures of the bacterial RNAP from other organisms include: QUANTA, CHARMM; INSIGHT; SYBYL; MACROMODE; and ICM.

A candidate drug can be selected by performing rational drug design with the three-dimensional structure determined for the crystal, preferably in conjunction with computer modeling discussed above. The candidate drug (e.g., a potential modulator of bacterial RNAP) can then be assayed as exemplified above, or in situ. A candidate drug can be identified as a drug, for example, if it inhibits bacterial proliferation.

A potential inhibitor (e.g., a candidate antibacterial agent) would be expected to interfere with bacterial growth. Therefore, an assay that can measure bacterial growth may be used to identify a candidate antibacterial agent.

Methods of testing a potential bacteriostatic or bacteriocidal compound (e.g., the candidate antibacterial agent) in isolated cultures and in animal models are well known in the art, and can include standard minimum-inhibitory-concentration (MIC) and minimum-bacteriocidal-concentration (MBC) assays. In animal models, the potential modulators can be administered by a variety of ways including topically, orally, subcutaneously, or intraperitoneally depending on the proposed use. Generally, at least two groups of animals are used in the assay, with at least one group being a control group, which is administered the administration vehicle without the potential modulator.

For all of the assays described herein further refinements to the structure of the compound generally will be necessary and can be made by the successive iterations of any and/or all of the steps provided by the particular screening assay.

It is anticipated that compounds identified according to the target and method of this invention would have applications not only in antibacterial therapy, as described above, but also in: (i) identification of bacterial RNAP (diagnostics, environmental-monitoring, and sensors applications), (ii) labeling of bacterial RNAP (diagnostics, environmental-monitoring, imaging, and sensors applications), (iii) immobilization of bacterial RNAP (diagnostics, environmental-monitoring, and sensors applications), (iv) purification of bacterial RNA polymerase (biotechnology applications), (v) regulation of bacterial gene expression (biotechnology applications), and (vi) antisepsis (antiseptics, disinfectants, and advanced-materials applications).

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

### EXAMPLES

### Example 1

### Cyclic Peptide MccJ25 (MccJ25) Inhibits Transcription by Binding within, and Obstructing, the RNAP Secondary Channel

Microcin J25 (MccJ25) is a 21 residue peptide antibiotic with an unusual, “lariat-protoknot” structure (Salomon and Farias (1992) J. Bacteriol. 174, 7428-7435; Bayro et al., (2003) J. Am. Chem. Soc. 125, 12382-12383; Rosengren et al., (2003) J. Am. Chem. Soc. 125, 12464-12474; Wilson et al., (2003) J. Am. Chem. Soc. 125, 12475-12483). MccJ25 is produced by Escherichia coli strains that harbor a plasmid-borne antibiotic-synthesis and antibiotic-export cassette, consisting of a gene for MccJ25 precursor (a 58 residue linear peptide), two genes for factors that process MccJ25 precursor into MccJ25, and one gene for export of MccJ25 (Solbiati et al., (1999) J. Bacteriol. 181, 2659-2662). MccJ25 exhibits bacteriocidal activity against a range of Gram-negative bacterial species, including E. coli.

Recently, it has been established that the functional target of MccJ25 is RNA polymerase (RNAP). Delgado et al. showed that MccJ25 inhibits transcription and identified a single-substitution MccJ25-resistant mutant of rpoC—the gene for the RNAP β′ subunit. (Delgado et al., (2001) J. Bacteriol. 183, 4543-4550). Yuzenkova et al. confirmed that MccJ25 inhibits transcription and identified six additional single-substitution MccJ25-resistant mutants of rpoC (two affecting the same codon as in the mutant of Delgado et al., 2001; four affecting other codons) (Yuzenkova et al. (2002) J. Biol. Chem. 277, 50867-50875).

The present invention provides that MccJ25 inhibits transcription in a purified system, provides the steps in transcription at which inhibition occurs, provides the mechanism by which inhibition occurs, and provides—through isolation of more than 120 independent single-substitution MccJ25-resistant mutants of rpoC following random and saturation mutagenesis—the determinant of RNAP for function of MccJ25. The present invention also provides that MccJ25 inhibits transcription by binding within, and obstructing, the RNAP secondary channel. This represents a novel mechanism for inhibition of a nucleotide polymerase and an attractive target for antibacterial drug discovery.

MccJ25 Does not Inhibit Open-Complex Formation:

Transcription Involves the Following Steps (Record et al., (1996) In Escherichia coli and Salmonella, F. C. Neidhart, ed. (Washington, D.C., ASM Press), pp. 792-820): (i) RNAP binds to promoter DNA, to yield an RNAP-promoter closed complex; (ii) RNAP melts approximately 14 bp of promoter DNA surrounding the transcription start site, to yield an RNAP-promoter open complex; (iii) RNAP begins synthesis of RNA, typically carrying out multiple rounds of abortive initiation (synthesis and release of RNA products less than 9-11 nt in length), as an RNAP-promoter initial transcribing complex; and (iv), upon synthesis of an RNA product of a critical threshold length of 9-11 nt, RNAP breaks its interactions with promoter DNA and begins to translocate along DNA, processively synthesizing RNA as an RNAP-DNA elongation complex.

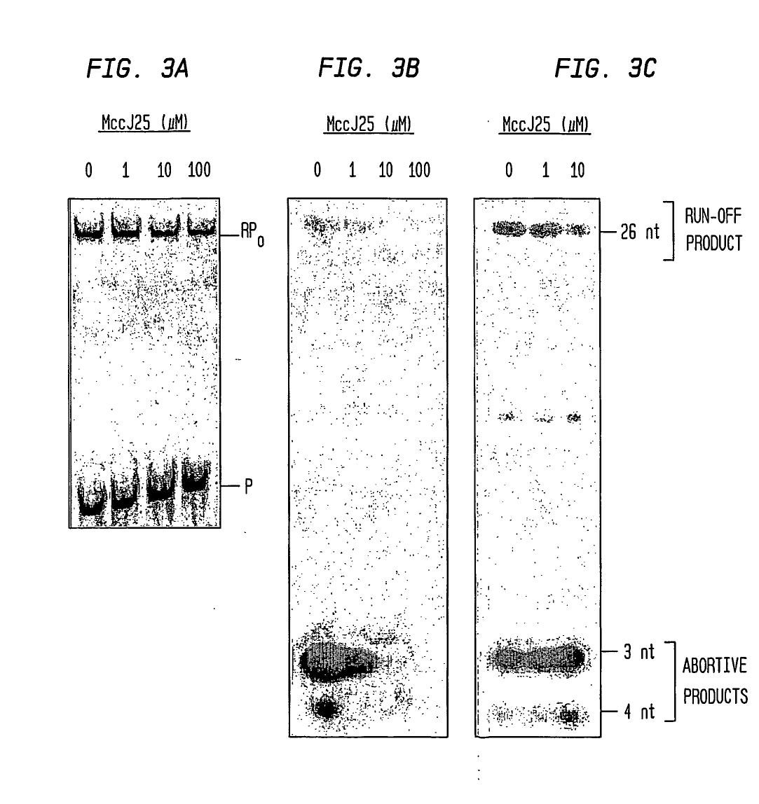

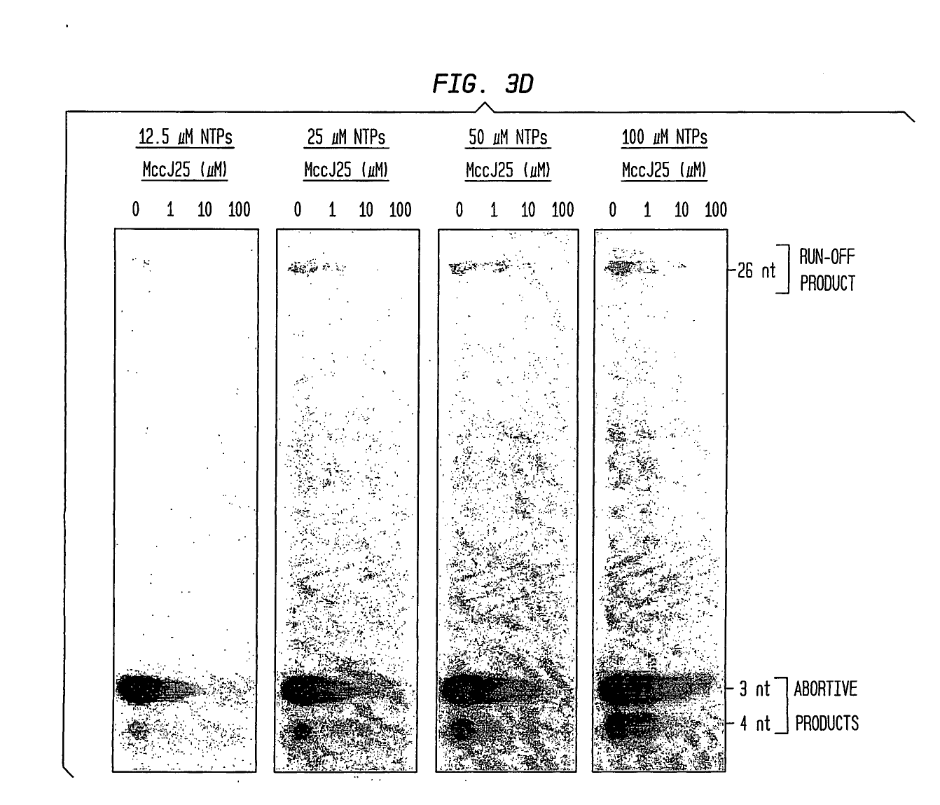

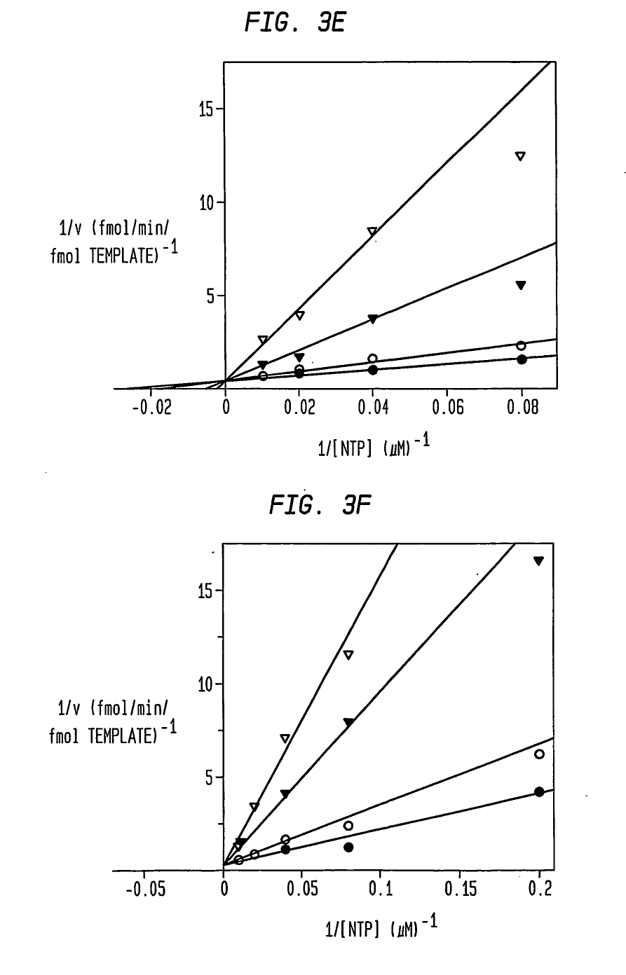

To determine whether MccJ25 inhibits steps in transcription up to and including formation of the RNAP-promoter open complex, electrophoretic mobility-shift experiments were performed. RNAP holoenzyme was incubated with a fluorochrome-labelled DNA fragment containing the lacUV5 promoter—in parallel in the absence and presence of MccJ25—and products were analyzed by non-denaturing PAGE followed by x/y fluorescence scanning (FIG. 3A). The results indicate that MccJ25 at a concentration of 1, 10, or 100 μM has no effect on formation of open complex (FIG. 3A). The results permit the conclusion that MccJ25 does not inhibit steps in transcription up to and including formation of open complex.

MccJ25 Inhibits Abortive Initiation and Elongation:

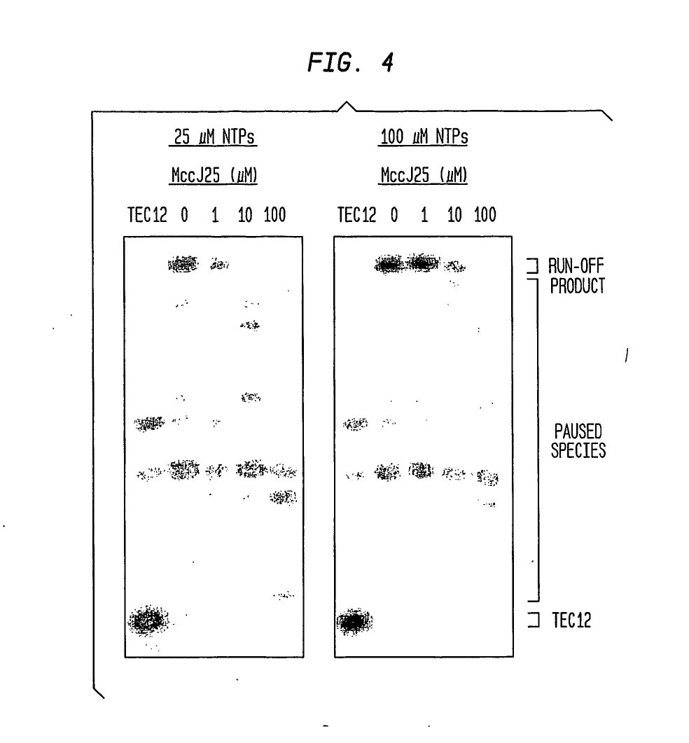

To determine whether MccJ25 inhibits steps in transcription subsequent to formation of the RNAP-promoter open complex, standard transcription experiments were performed. RNAP holoenzyme was pre-incubated with a DNA fragment containing the lacUVS promoter to form the RNAP-promoter open complex; radiolabelled NTPs were added and RNA synthesis was allowed to proceed for 5 min at 37° C.—in parallel in the absence and presence of MccJ25—and products were analyzed by urea-PAGE followed by storage-phosphor imaging (FIG. 3B). The results indicate that MccJ25 at a concentration of 1, 10, or 100 μM inhibits both formation of abortive products (3 nt and 4 nt RNA species produced in large stoichiometric excess over the DNA template; 86% inhibition at 100 μM MccJ25) and formation of the full-length product (26 nt RNA species; produced in stoichiometric equivalence with DNA template; 94% inhibition at 100 μM MccJ25) (FIG. 3B). The inhibition is specific; thus, inhibition is overcome completely upon substitution of residue 931 of the RNAP β′ subunit—the substitution shown by Delgado et al., (2001) to confer resistance to MccJ25 in vivo (FIG. 3C). Parallel experiments starting with a halted elongation complex, rather than with open complex, yield equivalent results with respect to inhibition of formation of full-length (FIG. 4). It is concluded that MccJ25 inhibits both abortive initiation and elongation. It is further concluded that MccJ25 interferes with a process common to both abortive initiation and elongation—i.e., NTP uptake, phosphodiester-bond formation, or translocation.

High Concentrations of NTPs Overcome Inhibition:

To assess whether MccJ25 Interferes with an NTP-concentration-dependent elementary reaction step, transcription experiments were performed at each of four NTP concentrations (12.5 μM, 25 μM, 50 μM, and 100 μM). The results indicate that high NTP concentrations can overcome inhibition by MccJ25 (FIG. 3D). As the NTP concentration increases, the extent of inhibition by MccJ25 decreases—both at the level of abortive products and at the level of the full-length product (FIG. 3D). It is concluded that MccJ25 interferes with an NTP-concentration-dependent elementary reaction step.

Inhibition is Partial Competitive with Respect to NTPs:

Quantitative analysis of the NTP-concentration-dependence data indicates that mode of inhibition by MccJ25 is partial competitive—i.e., that MccJ25 binds to a site on RNAP distinct from the NTP binding site and increases K_{M} for NTPs (FIG. 3E). (In conventional, or full, competitive inhibition, binding of the inhibitor and binding of the substrate are mutually exclusive (i.e., the inhibitor increases the apparent K_{M} for substrate to infinity) (Segel, (1975) Enzyme Kinetics (New York, Wiley). In contrast, in partial competitive inhibition, binding of the inhibitor and binding of the substrate are mutually inhibitory, but not mutually exclusive (i.e., the inhibitor increases the apparent K_{M} for substrate, but does not increase apparent K_{M} for substrate to infinity) (Segel, (1975)). Both full and partial competitive inhibition yield double-reciprocal plots that intersect at the y-axis; however, full and partial competitive inhibition yield double-reciprocal plots with different dependences of slope on inhibitor concentration and thus readily can be distinguished. Partial competitive inhibition implies that the inhibitor site and substrate site on the enzyme must be distinct, in part or in whole. Kᵢ, the reciprocal of the equilibrium binding constant for MccJ25-RNAP interaction, is estimated to be 1.4±0.2 μM; α, the factor by which MccJ25 increases K_{M} for NTPs, is estimated to be 15±3 (FIG. 3E). Fluorescence-detected abortive initiation assays assessing iterative tri- and tetranucleotide synthesis—assays for which the initial-velocity assumption is rigorously valid—yield equivalent results: i.e., partial-competitive inhibition, with Kᵢ=1.2±0.3 μM, and α 8.7±2 (FIG. 3F). It is concluded that MccJ25 inhibits transcription by binding to a site on RNAP distinct from the NTP binding site and interfering with NTP uptake.

MccJ25 Requires an Extensive Determinant within the RNAP Secondary Channel:

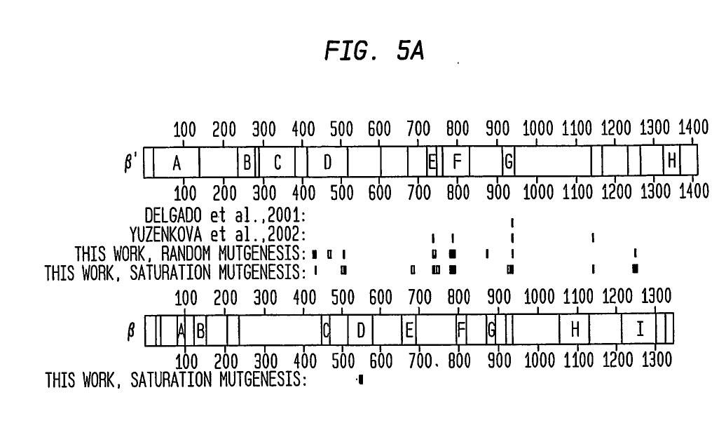

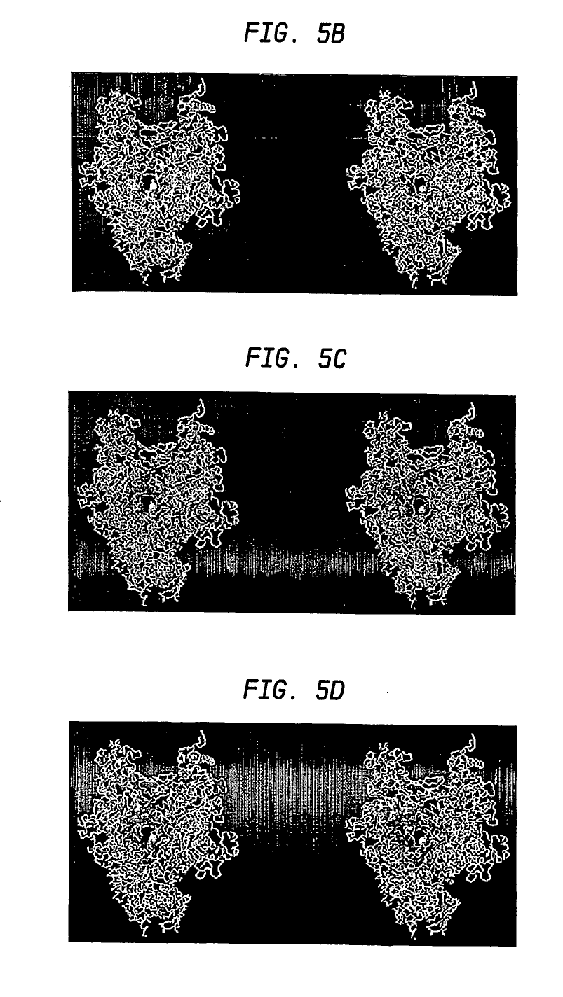

The MccJ25-resistant rpoC mutant of Delgado et al. 2001 results in substitution of residue 931 of RNAP β′ subunit (Thr931→Ile). In the primary structure of β′, residue 931 maps to conserved region G (FIG. 5A). In the three-dimensional structure of bacterial RNAP, residue 931 of β′ maps to the RNAP NTP-uptake channel (also referred to as the “secondary channel,” “nucleotide-uptake channel” or “pore”; FIG. 5B) The MccJ25-resistant rpoC mutants of Yuzenkova et al., 2002 also map to the RNAP secondary channel (FIG. 5B). The RNAP secondary channel is an ˜30 Å long, ˜10-15 Å wide, tunnel that connects the exterior surface of RNAP with the RNAP active center (Zhang et al., (1999) Cell 98, 811-824; Cramer et al., (2001) Science 292, 1863-1876). According to generally accepted models, NTPs must pass through the RNAP secondary channel in order to access the RNAP active-center and NTP binding site (Zhang et al., 1999; Cramer et al., 2000; Korzheva et al., (2000) Science 289, 619-625; Gnatt et al., (2001) Science 292, 1876-1882). Thus, the location of the substitutions, in conjunction with the finding above that MccJ25 inhibits transcription by interfering with NTP uptake, immediately suggests a possible mechanism of inhibition: i.e., MccJ25 may inhibit transcription by binding within, and obstructing, the RNAP secondary channel.

To define determinants of β′ specifically required for transcription inhibition by MccJ25—and thereby to test the hypothesis that determinants for binding of MccJ25 are located within the RNAP NTP-uptake channel—random mutagenesis of the entire gene encoding β′ was performed and MccJ25-resistant mutants were isolated and characterized. (RNAP β′ subunit comprises ˜50% of all residues of RNAP and comprises ˜90% of all residues of the RNAP secondary channel.) Mutagenesis using error-prone PCR (Zhou et al., (1991) Nucl. Acids Res. 19, 6052) of five DNA segments spanning the length of a plasmid-borne rpoC gene were performed (Table 1). Overall, 20 mutagenesis reactions were performed, ˜100,000 candidates were analyzed, and 22 independent plasmid-linked MccJ25-resistant mutants were isolated (Table 1). Minimum-bacteriocidal-concentration (MBC) assays indicate that all 22 MccJ25-resistant mutants exhibit ≧5-fold increases in MBC, and 18 of 22 MccJ25-resistant mutants exhibit ≧50-fold increases in MBC (Table 2, column 5). Complementation assays indicate that all 22 MccJ25-resistant mutants can complement an rpoC^{ts} mutant for growth at the non-permissive temperature, confirming that each encodes a β′ derivative functional in transcription—indeed sufficiently functional in transcription to support viability (Table 2, column 4).

For each of the 22 MccJ25-resistant mutants, the DNA-nucleotide sequence of the relevant segment of the rpoC gene was determined, and the amino acid sequence of the substituted β′ derivative was inferred (Table 2). Nineteen different substitutions, involving eighteen different sites within β′, were obtained (Table 2).

TABLE 1
Summary of random mutagenesis and selection

| codons 1-292 | |
|---|---|
| mutagenesis reactions | 4 |
| total candidates | 11,000 |
| independent plasmid-linked MccJ25^{r} candidates | 0 |
| codons 292-546 | |
| mutagenesis reactions | 3 |
| total candidates | 13,000 |
| independent plasmid-linked MccJ25^{r} candidates | 9 |
| codons 546-876 | |
| mutagenesis reactions | 4 |
| total candidates | 11,000 |
| independent plasmid-linked MccJ25^{r} candidates | 11 |
| codons 876-1213 | |
| mutagenesis reactions | 5 |
| total candidates | 36,000 |
| independent plasmid-linked MccJ25^{r} candidates | 1 |
| codons 1213-1408 | |
| mutagenesis reactions | 4 |
| total candidates | 25, 000 |
| independent plasmid-linked MccJ25^{r} candidates | 1 |
| overall | |
| mutagenesis reactions | 20 |
| total candidates | 96,000 |
| independent plasmid-linked MccJ25^{r} candidates | 22 |

TABLE 2
MccJ25^{r} isolates from random mutagenesis and selection

| | | | | |
|---|---|---|---|---|
| | | Number of | | |
| Amino acid | Codon | independent | Complementation | MBC* |
| substitution | substitution | isolates | of rpoC^{ts} | (mg/ml) |

| | | | | | |
|---|---|---|---|---|---|
| None | | none | — | ++ | 0.0 |
| | | | | | 1 |
| 424 | Asn→Ser | AAC→AGC | 1** | + | 0.1 |
| 428 | Thr→Ile | ACT→ATT | 2 | ++ | 0.5 |
| 430 | His→Leu | CAC→CTC | 3 | + | 0.5 |
| 464 | Asp→Gly | GAC→GCC | 1 | ++ | 0.0 |
| 469 | His→Arg | CAC→CGC | 1 | ++ | 0.0 |
| 504 | Gln→Arg | CAG→CGG | 1 | ++ | 0.5 |
| 733 | Ser→Phe | TCT→TTC | 1 | + | 0.5 |
| 738 | Arg→Leu | CGT→CTT | 1 | ++ | 0.0 |
| 776 | Thr→Ile | ACC→ATC | 1 | ++ | 0.5 |
| 779 | Ala→Thr | GCT→ACT | 1 | ++ | 1 |
| 780 | Arg→Cys | CGT→TGT | 1 | ++ | 1 |
| 782 | Gly→Ala | GGT→GCT | 1 | ++ | 1 |
| 785 | Asp→Gly | GAT→GGT | 1 | ++ | 0.5 |
| 786 | Thr→Ile | ACC→ATC | 1 | ++ | 0.5 |
| 789 | Lys→Arg | AAA→AGA | 1 | ++ | 0.5 |
| 789 | Lys→Gln | AAA→CAA | 1 | ++ | 1 |
| 869 | Cys→Arg | TGT→CGT | 1 | + | 1 |
| 933 | Arg→Cys | CGT→TGT | 1 | ++ | 1 |
| 1244 | Gln→Leu | CAG→CTG | 1 | ++ | 0.5 |

*Minimum bacteriocidal concentration; defined as the lowest concentration of NccJ25 that yields a viable cell count of ⁻ after incubation 2 h at 37° C. (see Experimental Procedures).
**Isolated as double mutant 354 Val→Ile; 424 Asn→Ser; complementation and MBC data are for a single mutant constructed using site-directed mutagenesis.

In the primary structure of β′, the sites at which substitutions were obtained map to conserved region D, conserved region E, conserved region F, the segment between conserved regions F and G, conserved region G, and the segment between conserved regions G and H (referred to as conserved region G′ by Zakharova et al., (1998) J. Biol. Chem. 273, 24912-24920) (FIG. 5A). In the three-dimensional structure of RNAP, the locations of the substitutions are tightly clustered—and are centered on the RNAP secondary channel (FIG. 5C). The substitutions, without exception, map to residues that line the RNAP secondary channel, or to residues that make direct contact with residues that line the RNAP secondary channel (FIG. 5C). The substitutions map to the floor, the roof, and the walls of RNAP secondary channel (FIG. 5C). It is concluded that the RNAP secondary channel contains a multi-residue determinant for function of MccJ25. Based on the fact that substitutions conferring MccJ25-resistance were obtained at none of the >1000 residues of β′ outside the immediate vicinity of the secondary channel, it is further concluded that no part of β′ outside the immediate vicinity of the secondary channel contains a determinant for function of MccJ255.

MccJ25 Requires More than Fifty Residues that Line the RNAP Secondary Channel:

To define systematically the MccJ25 determinant within the RNAP secondary channel, saturation mutagenesis was performed of the rpoC gene (encoding β′) and the rpoB gene (encoding β), targeting all codons for residues that line the RNAP secondary channel. Saturation mutagenesis was performed using a set of eleven “doped” oligodeoxyribonucleotide primers, designed to introduce all possible nucleotide substitutions at all positions of all codons for residues that line the RNAP secondary channel (sequences in Table 3; methods as in Ner et al., (1988) DNA 7, 127-134; Hermes et al., (1989) Gene 84, 143-151). In total, 25 mutagenesis reactions were performed, ˜40,000 candidates were analyzed, and 114 independent plasmid-linked MccJ25-resistant mutants were isolated and characterized (Table 4).

TABLE 3

Sequences of “doped” oligonucleotide primers
used in saturation mutagenesis

| | |
|---|---|
| Codon | Sequence |

| | |
|---|---|
| rpoC | |
| 429-433* | CCGTGCACCGACTCTGCACCGTCTGGGTATCCAGGCATT |
| | TG |
| 492-504** | GCAACAACATCCTGTCCCCGGCGAACGGCGAACCAATCA |
| | TCGTTCCGTCTCAGGACGTTGTACTGGGTC |
| 597-603* | GTCAACCAGGCGCTGGGTAAAAAAGCAATCTCCAAAATG |
| | CTGAACACCTGC |
| 680-698** | CGGGCGAACGCTACAACAAAGTTATCGATATCTGGGGCT |
| | GCGGCGAACGATCGTGTATCCAAAGCGATGATGGATAAC |
| | CTGCAAAC |
| 726-740** | CTACATGATGGCCGACTCCGGTGCGCGTGGTTCTGCGGC |
| | ACAGATTCGTCAGCTTGCTGGTATG |
| 741-754** | CGTCAGCTTGCTGGTATGCGTGGTCTGATGGCGAAGCCG |
| | GATGGCTCCATCATCGAAACG |
| 775-790** | GTACTTCATCTCCACCCACGGTGCTCGTAAAGGTCTGGC |
| | GGATACCGCACTGAAAACTGCGAACTCCG |
| 922-933** | GTGTTATCGCGGGACAGTCCATCGGTGAACCGGGTACAC |
| | AGCTGACCATGCGTACGTTCCACATCGGTGG |
| 1136-1137* | CCAAGGACATCACCGGTGGTCTGCCGCGCGTTGC |
| 1239-1248** | CGAAGTACAGGACGTATACCGTGTGCAGGGCGTTAAGAT |
| | TAACGATAAAC |
| rpoB | |
| 542-549** | CTGACCCGTGAACGTGCAGGCTTCGAAGTTCGAGACGTA |
| | CACCCG |

*Underlined nucleotides were synthesized using phosphoramidite reservoirs having 92% correct phosphoramidite and 8% of a 1:1:1:1 mix of dA, dC, dG, and dT phosphoramidites (i.e., 94% of total correct phosphoramidites and 6% of total incorrect phosphoramidites).
**Underlined nucleotides were synthesized using phosphoramidite reservoirs having 98% correct phosphoramidite and 2% of a 1:1:1:1 mix of dA, dC, dG, and dT phosphoramidites (i.e., 98.5% of total correct phosphoramidites and 1.5% of total incorrect phosphoramidites).

TABLE 4
Summary of saturation mutagenesis and selection

| rpoC | |
|---|---|
| codons 429-433 | |
| total candidates | 6,000 |
| plasmid-linked MccJ25^{r} candidates | 8 |
| codons 492-504 | |
| total candidates | 2,000 |
| plasmid-linked MccJ25^{r} candidates | 9 |
| codons 597-603 | |
| total candidates | 5,000 |
| plasmid-linked MccJ25^{r} candidates | 0 |
| codons 680-698 | |
| total candidates | 8,000 |
| plasmid-linked MccJ25^{r} candidates | 6 |
| codons 726-740 | |
| total candidates | 3,000 |
| plasmid-linked MccJ25^{r} candidates | 12 |
| codons 741-754 | |
| total candidates | 4,000 |
| plasmid-linked MccJ25^{r} candidates | 5 |
| codons 775-790 | |
| total candidates | 3,000 |
| plasmid-linked MccJ25^{r} candidates | 33 |
| codons 922-933 | |
| total candidates | 1,000 |
| plasmid-linked MccJ25^{r} candidates | 13 |
| codons 1136-1137 | |
| total candidates | 2,000 |
| plasmid-linked MccJ25^{r} candidates | 4 |
| codons 1239-1248 | |
| total candidates | 3,000 |
| plasmid-linked MccJ25^{r} candidates | 15 |
| rpoB | |
| codons 542-549 | |
| total candidates | 3,000 |
| plasmid-linked MccJ25^{r} candidates | 9 |

Sequencing indicates that 106 of the 114 MccJ25-resistant mutants are single-substitution mutants (Table 5). The single-substitution mutants comprise 71 different substitutions, involving 43 different sites within β′ (Table 5). Taken together, the single-substitution mutants from random mutagenesis and saturation mutagenesis comprise 80 different substitutions, involving 47 different sites within β′ and 4 different sites within β (Tables 2, 5).

TABLE 5
MccJ25^{r} isolates from Saturation mutagenesis and

| | | Number of | |
|---|---|---|---|
| Amino acid | | independent | MBC* |
| Substitution | | isolates | (mg/ml) |
| rpoC | | | |
| single-substitution mutants | | | |
| 428 | Thr→Ile | 3 | 0.5 |
| 428 | Thr→Asn | 2 | 0.5 |
| 429 | Leu→Gln | 2 | 0.5 |
| 430 | His→Tyr | 1 | 0.5 |
| 498 | Pro→Leu | 1 | 1 |
| 498 | Pro→Gln | 1 | 1 |
| 503 | Ser→Pro | 1 | 0.5 |
| 503 | Ser→Tyr | 1 | 0.5 |
| 504 | Gln→Arg | 1 | 0.5 |
| 504 | Gln→Glu | 2 | 0.5 |
| 508 | Leu→Val | 1 | 0.05 |
| 680 | Asn→Lys | 2 | 0.5 |
| 684 | Asp→Ala | 1 | 1 |
| 684 | Asp→Tyr | 1 | 0.5 |
| 684 | Asp→Glu | 1 | 0.5 |
| 684 | Asp→Val | 1 | 0.5 |
| 732 | Gly→Asp | 2 | 0.1 |
| 733 | Ser→Phe | 1 | 0.1 |
| 733 | Ser→Val | 1 | 0.5 |
| 733 | Ser→Tyr | 1 | 0.1 |
| 735 | Ala→Δ | 2 | 1 |
| 736 | Gln→Pro | 1 | 0.5 |
| 738 | Arg→Leu | 1 | 0.05 |
| 744 | Arg→Pro | 1 | 0.05 |
| 744 | Arg→His | 1 | 0.05 |
| 748 | Ala→Pro | 3 | 0.1 |
| 775 | Ser→Cys | 1 | 0.5 |
| 776 | Thr→Ile | 3 | 0.5 |
| 777 | His→Tyr | 1 | 1 |
| 777 | His→Pro | 2 | 0.5 |
| 779 | Ala→Gly | 1 | 0.5 |
| 779 | Ala→Thr | 1 | 1 |
| 779 | Ala→Pro | 2 | 0.5 |
| 780 | Arg→Cys | 1 | 1 |
| 782 | Gly→Ala | 1 | 1 |
| 783 | Leu→Pro | 1 | 0.1 |
| 784 | Ala→Glu | 1 | 0.5 |
| 785 | Asp→Gly | 2 | 0.5 |
| 786 | Thr→Ile | 1 | 0.5 |
| 786 | Thr→Ala | 2 | 0.5 |
| 788 | Leu→Met | 1 | 1 |
| 789 | Lys→Asn | 2 | 1 |
| 789 | Lys→Gln | 1 | 1 |
| 789 | Lys→Arg | 1 | 0.5 |
| 790 | Thr→Ala | 2 | 1 |
| 790 | Thr→Ile | 2 | 1 |
| 790 | Thr→Ser | 2 | 0.5 |
| 790 | Thr→Asn | 1 | 0.5 |
| 922 | Cys→Tyr | 1 | 0.5 |
| 926 | Pro→Ser | 1 | 0.5 |
| 927 | Gly→Ser | 2 | 1 |
| 927 | Gly→Cys | 1 | 1 |
| 930 | Leu→Met | 1 | 1 |
| 931 | Thr→Ile | 2 | 1 |
| 931 | Thr→Ala | 2 | 1 |
| 932 | Met→Ile | 2 | 1 |
| 933 | Arg→Cys | 1 | 1 |
| 1136 | Gly→Cys | 1 | 1 |
| 1136 | Gly→Ala | 1 | 0.5 |
| 1137 | Gly→Ala | 1 | 0.5 |
| 1137 | Gly→Arg | 1 | 1 |
| 1240 | Val→Glu | 1 | 0.5 |
| 1241 | Tyr→Ser | 2 | 0.1 |
| 1241 | Tyr→His | 2 | 0.1 |
| 1241 | Tyr→Cys | 1 | 0.1 |
| 1244 | Gln→Pro | 2 | 0.1 |
| 1244 | Gln→Leu | 1 | 0.5 |
| 1244 | Gln→Glu | 2 | 0.1 |
| 1247 | Lys→Glu | 2 | 0.1 |
| 1247 | Lys→Gln | 1 | 0.1 |
| 1248 | Ile→Ser | 1 | 0.1 |
| multiple-substitution mutants | | | |
| 493 | Pro→Thr; 498 | 1 | 0.1 |
| | Pro→Thr | | |
| 732 | Gly→Asp; 733 | 1 | 0.1 |
| | Ser→Ala | | |
| 732 | Gly→Asp; | 1 | 0.5 |
| | Gly→Thr | | |
| 733 | Ser→Val; 734 | 1 | 0.1 |
| | Ala→Gly | | |
| 777 | His→Ser; 778 | 1 | 0.5 |
| | Gly→Ala | | |
| rpoB | | | |
| single-substitution mutants | | | |
| 544 | Gly→Asp | 1 | 0.05 |
| 545 | Phe→Ser | 2 | 0.05 |
| 548 | Arg→Gln | 2 | 0.05 |
| 549 | Asp→His | 1 | 0.05 |
| multiple-substitution mutants | | | |
| 543 | Ala→Pro; 546 | 1 | 0.05 |
| | Glu→Gly | | |
| 543 | Ala→Leu; 549 | 1 | 0.05 |
| | Asp→Tyr | | |
| 545 | Phe→Leu; 546 | 1 | 0.05 |
| | Glu→Asp | | |

Minimum bacteriocidal concentration; defined as the lowest concentration of MccJ25 that yields a viable cell count of ˜0 after incubation 2 h at 37° C. (see Experimental Procedures).

In the three-dimensional structure of RNAP, the sites at which single substitutions were obtained define a nearly continuous surface, comprising most of the interior lining and part of the rim of the RNAP secondary channel (FIG. 5D). The sites span nearly the full circumference of the RNAP secondary channel (FIG. 5D). The side chains of the majority of implicated residues are solvent-accessible—directed into the lumen of the RNAP secondary channel or toward the exterior of RNAP—and make no obvious interactions important for RNAP structure or function.

It is concluded that the RNAP secondary channel contains an extensive determinant for function of MccJ25. Based on the size of the determinant (nearly 50 residues; Tables 2 and 5), the architecture of the determinant (interior of hollow cylinder), and the solvent-accessibility of the determinant, it is concluded that the determinant corresponds to the binding site on RNAP for MccJ25. For reference, the size and molecular mass of MccJ25 (2,107 Da; diameter=19 Å, modeled as a sphere) would allow MccJ25 to make direct contact with all, or nearly all, residues of the determinant. It further is concluded that the sites of substitutions that confer MccJ25-resistance map the binding site on RNAP for MccJ25 and, in essence, serve as a genetic footprint of the binding site.

Thirteen of fifteen sites associated with the highest level of MccJ25-resistance (MBC=1 mg/ml; Tables 2 and 5) cluster in an ˜20 Å×˜20 Å×˜20 Å sub-region of the RNAP secondary channel, bounded by the α-helix containing residue 684 (D/E helix), the α-helix containing residue 735 (E helix), the α-helix containing residues 777-790 (F helix), the α-helix and loop containing residues 927-933 (G helix and loop), and loop containing residues 1136-1137 (G′ loop) (sub-region above and to left of the active-center Mg⁺⁺ in view in FIG. 5D). It is concluded that this sub-region is the most important part of the determinant.

The present invention provides that MccJ25 inhibits the abortive-initiation and elongation phases of transcription, that inhibition involves interference with NTP uptake or NTP binding, and that inhibition is partial-competitive with respect to NTPs (i.e., involves a site distinct from the RNAP NTP binding site). The present invention further provides that inhibition involves an extensive determinant within the RNAP secondary channel, comprising nearly the entire lining of the RNAP secondary channel (more than fifty sites for substitutions conferring MccJ25-resistance). The present invention further provides that MccJ25 inhibits transcription by interfering with NTP uptake by binding within, and obstructing, the RNAP secondary channel—acting essentially as a “cork in a bottle.”

The present invention also provides that a molecule the size of MccJ25 (2.1 kDa; diameter=19 Å, modeled as a sphere) readily could bind within, and obstruct, the RNAP secondary channel (dimensions=25 Å by 10-15 Å).

The present invention also provides that obstruction of the RNAP secondary channel represents a novel mechanism of inhibition of RNAP. Rifampicin inhibits bacterial RNAP by sterically preventing synthesis of an RNA product longer than ˜4 nt (Campbell et al., (2001) Cell 104, 901-912). Streptolydigin, arylhydroxamidines, and pyrazoles are proposed to inhibit bacterial RNAP through interference with active-center conformational changes associated with phosphodiester-bond formation and/or translocation (Epshtein et al., (2002) Mol. Cell. 10, 623-634; Artsimovitch et al., (2003) Science 302, 650-654). α-Amanitin is proposed to inhibit eukaryotic RNAP II through interference with active-center conformational changes associated with phosphodiester-bond formation and/or translocation (Bushnell et al., (2002) Proc. Natl. Acad. Sci. USA 99, 1218-1222). The present invention also provides that obstruction of the RNAP secondary channel represents an exceptionally attractive target for development of novel antibacterial agents. First, the RNAP secondary channel is eminently “druggable,” presenting an extended, encircling surface complementary to a range of molecules—like MccJ25—that have molecular weights of 500-2,500 Da. Second, the RNAP secondary channel exhibits distinct patterns of sequence conservation in bacterial RNAP and eukaryotic RNAP, permitting identification of agents—like MccJ25—that inhibit bacterial RNAP but do not inhibit eukaryotic RNAP. Third, the RNAP secondary channel is distinct from, and well-separated from, the binding site of the RNAP inhibitor in current use in antibacterial therapy, rifampicin, permitting identification of agents—like MccJ25 (E. S. and R. H. E., unpublished data)—that do not exhibit cross-resistance with rifampicin.

Experimental Procedures:

Plasmids:

Plasmid pTUC202 carries genes for synthesis and export of MccJ25 (Solbiati et al., (1996)). Plasmid pRL663 encodes C-terminally hexahistidine-tagged E. coli RNAP β′ subunit under control of the tac promoter (Wang et al., (1995) Cell 81, 341-350). Plasmid pRL706 encodes C-terminally hexahistidine-tagged E. coli RNAP β subunit under control of the trc promoter (Severinov et al., (1997) J. Biol. Chem. 272, 24137-24140). Plasmid pREII-NHα encodes N-terminally hexahistidine-tagged E. coli RNAP α subunit under control of tandem lpp and ′lacUV5 promoters (Niu et al., (1996) Cell 87, 1123-1134).

MccJ25:

MccJ25 was purified essentially as in Blond et al., (1999) Eur. J. Biochem. 259, 747-755. E. coli strain DH5α [hsdR17 recA1 relA1 endA1 gyrA96 gal deoR phoA supE44 thi Δ(lacZYA-argF)U169 φ80 dlacZΔM15; Invitrogen, Inc.] transformed with plasmid pTUC202 (Solbiati et al., (1996)) was shaken in 1 L M9 medium (Sambrook and Russel, (2001) Molecular Cloning (Cold Spring Harbor, N.Y., Cold Spring Harbor Laboratory)) for 24 h at 37° C., and the culture medium was harvested by centrifugation (5000×g; 15 min at 22° C.). The culture medium was applied to a preparative C8 cartridge (10 g Mega-BE C8; Varian, Inc.); the cartridge washed successively with 50 ml water, 50 ml 20% methanol, and 50 ml 50% methanol; the cartridge was eluted with 4×5 ml fractions of 80% methanol; and fractions containing MccJ25 (detected by UV absorbance at 276 nm) were pooled, lyophilized, re-dissolved in 10′ ml 20% methanol, and stored in aliquots at −20° C. Yields typically were 100 mg [determined by UV-absorbance at 276 nm using ε₂₇₆=2,900 M⁻¹ cm⁻¹ (calculated as in Gill and von Hippel, (1989) Anal. Biochem. 182, 319-326)]. Purities typically were 50% (determined by analytical reversed-phase HPLC). Samples for electrophoretic mobility shift assays, transcription assays, and fluorescence-detected abortive initiation assays were further purified by reversed-phase HPLC on a C18, 5 μM, 300 Å column (Rainin, Inc.), with solvent A=20% methanol and 0.1% trifluoroacetic acid, solvent B=methanol and 0.1% trifluoroacetic acid, gradient=0-90% solvent B in solvent A in 50 min, and flow rate=1 ml/min. Fractions containing MccJ25 (retention time ˜30 min; detected by UV absorbance at 276 nm) were pooled, lyophilized, re-dissolved in 500 μl 10% methanol, and stored in aliquots at −20° C. Yields typically were 50 mg per 1 L culture. Purities typically were >95%.)

RNAP:

RNAP core and holoenzyme were prepared as in Niu et al., (1996). [Ile931]β′ RNAP core and holoenzyme were prepared from strain 397c [rpoC^{ts}397 argG thi lac (λcI₈₅₇h₈₀Sₜ₆₈dlac⁺); Christie et al., (1996) J. Bacteriol. 178, 6991-6993] transformed with a pRL663 derivative encoding [Ile931] β′-RNAP using analogous procedures. Yields typically were 6 mg. Purities typically were >95%.

Electrophoretic Mobility Shift Assays:

Reaction mixtures contained (20 μl): 100 nM RNAP holoenzyme, 20 nM DNA fragment lacUV5-12(Cy5,+26) (Mukhopadhyay et al., (2001) Cell 106, 453-463), and 0-100 μM MccJ25 in TB [50 mM Tris-HCl (pH 8.0), 100 mM KCl, 10 mM MgCl₂, 1 mM dithiothreitol, 10 μg/ml bovine serum albumin, and 5% glycerol]. Following 15 min at 37° C., 0.5 μl 1 mg/ml heparin was added (to disrupt non-specific complexes), and, following a further 2 min at 37° C., reaction mixtures were applied to 5% polyacrylamide slab gels (30:1 acrylamide/bisacrylamide; 6×9×0.1 cm) and electrophoresed in 90 mM Tris-borate (pH 8.0) and 0.2 mM EDTA (20 V/cm; 30 min at 37° C.) and analyzed using a fluorescence scanner (Storm 860; Molecular Dynamics)

Transcription Assays:

Reaction mixtures contained (18 μl): 100 nM RNAP holoenzyme, 20 nM DNA fragment lacUV5-12(Cy5,+26) (Mukhopadhyay et al., (2001)), and 0-100 μM MccJ25 in TB. Following 15 min at 37° C., 0.5 μl 1 mg/ml heparin was added, and, following a further 2 min at 37° C., RNA synthesis was initiated by addition of 2 μl 5 mM ApA and 125 μM (or 250 μM, 500 μM, and 1 mM) each of [α-³²P]UTP (0.6 Bq/fmol), ATP, CTP, GTP. Following 5 min at 37° C., reactions were terminated by addition of 10 μl 80% formamide, 10 mM EDTA, 0.04% bromophenol blue, and 0.04% xylene cyanol. Products were heated 10 min at 90° C., resolved by urea-PAGE (Sambrook and Russel, (2001)), and quantified using a storage-phosphor scanner (Storm 860; Molecular Dynamics). Identities of tri- and tetranucleotide abortive products were defined as in Borowiec and Gralla, (1985) J. Mol. Biol. 184, 587-598). Data were fit to full-competitive, partial-competitive, full-noncompetitive, partial-noncompetitive, full-uncompetitive, partial-uncompetitive, full-mixed, and partial-mixed models of inhibition using the Fit-To-Model feature of the SigmaPlot Enzyme Kinetics Module (SPSS).

Fluorescence-Detected Abortive Initiation Assays:

Reaction mixtures contained (46.5 μl): 100 nM RNAP holoenzyme, 20 nM DNA fragment lacUV5-12 (Mukhopadhyay et al., (2001)), and 0-100 μM MccJ25 in TB. Following 15 min at 37° C., 0.5 μl 1 mg/ml heparin was added, and, following a further 2 min at 37° C., 1 μl of 0.25-5 mM (γ-AmNS)UTP (Molecular Probes) was added, and reaction mixtures were transferred to sub-micro fluorometer cuvettes (Starna Cells). Following 2 min at 37° C., RNA synthesis was initiated by addition of 2.5 μl 10 mM AₚA, and fluorescence emission intensity was monitored 5 min at 37° C. [excitation wavelength=360 nm and emission wavelength=500 nm; excitation and emission slit widths=4 nm; QuantaMaster QM1 spectrofluorometer (PTI)]. The quantity of UMP incorporated into RNA was determined from the quantity of (γ-AmNS)UTP consumed, which, in turn, was calculated as follows (Schlageck et al., (1979) J. Biol. Chem. 254, 12074-12077):
(γ-AmNS)UTP_{consumed}=[(γ-AmNS)UTP₀](Fₜ−F₀)/(12.4×F₀) (1)
where (γ-AmNS)UTP₀ is the quantity of (γ-AmNS) UTP at time 0, F₀ is the fluorescence emission intensity at time 0, and Fₜ is the fluorescence emission intensity at time t. Data were fit to full-competitive, partial-competitive, full-noncompetitive, partial-noncompetitive, full-uncompetitive, partial-uncompetitive, full-mixed, and partial-mixed models of inhibition as in the preceding section.
Random Mutagenesis:

Random mutagenesis was performed by error-prone PCR amplification of the XbaI-SnaBI (codons 1-292), SnaBI-SphI (codons 292-546), SphI-SalI (codons 546-876), SalI-BspEI (codons 876-1213), and BspEI-XhoI (codons 1213-1408) rpoC segments of plasmid pRL663 (procedure of Zhou (1991) Nucl. Acids Res. 19, 6052; Zhou et al., (1993) Proc. Natl. Acad. Sci. USA 90, 6081-6085). The mutagenesis procedure yields all possible transition and transversion substitutions (Id. at Table 5). Mutagenized plasmid DNA was introduced by transformation into strain Stb12 [mcrA Δ(mcrBC-hsdRMS-mrr) recA1 relA1 endA1 gyrA96 lon supE44 thi Δ(lac-proAB); Invitrogen, Inc.], transformants (−10⁴ cells) were applied to LB-agar plates (Sambrook and Russell (2001)) containing 1 μg/ml MccJ25 and 200 μg/ml ampicillin, and plates were incubated 24 h at 37° C. followed by 0-48 h at 25° C. For each MccJ25^{r} clone (identified as a clone yielding a colony on the original selective plate and also yielding colonies when re-streaked to the same medium and incubated 16 h at 37° C.), plasmid DNA was prepared, plasmid DNA was introduced by transformation into strain DH5α [hsdR17 recA1 relA1 endA1 gyrA96 gal deoR phoA supE44 thi Δ(lacZYA-argF)U169 ø80dlacZΔM15; Invitrogen, Inc.], transformants (˜10⁴ cells) were applied to LB-agar plates containing I μg/ml MccJ25 and 200 μg/ml ampicillin and, in parallel, to LB-agar plates containing 200 μg/ml ampicillin, and plates were incubated 16 h at 32° C. For each plasmid-linked MccJ25r clone (identified as a clone yielding comparable numbers of colonies on the plates with and without MccJ25), the nucleotide sequence of the mutagenized rpoC segment was determined by dideoxy nucleotide sequencing.

Saturation Mutagenesis:

A set of “doped” oligodeoxyribonucleotide primers corresponding to codons 425-437, 487-509, 592-608, 675-703, 723-743, 739-757, 772-793, 918-937, 1132-1141, and 1236-1251 of the rpoC gene of pRL663 was synthesized on an AB392 automated synthesizer (Applied Biosystems, Inc). using solid-phase β-cyanoethylphosphoramidite chemistry (sequences in Table 4). The level of “doping” (nucleotide misincorporation) was selected to yield an average of 0.4-1 substitution per molecule of oligodeoxyribonucleotide primer (equations in Hermes et al., (1989)). Thus, the nucleotides corresponding to codons 429-433, 597-603, and 1136-1137 were synthesized using phosphoramidite reservoirs containing 92% of the correct phosphoramidite and 8% of a 1:1:1:1 mix of dA, dC, dG, and dT phosphoramidites (i.e., 94% total correct phosphoramidite and 6% total incorrect phosphoramidite); the nucleotides corresponding to codons 492-504, 680-698, 726-740, 74 1-754, 775-790, 922-933, and 123 9-1248 were synthesized using phosphoramidite reservoirs containing 98% of the correct phosphoramidite and 2% of a 1:1:1:1 mix of dA, dC, dG, and dT phosphoramidites (i.e., 98.5% total correct phosphoramidite and 1.5% total incorrect phosphoramidite); and all other nucleotides were synthesized using phosphoramidite reservoirs containing 100% of the correct phosphoramidite. Primer-extension mutagenesis reactions were performed using the QuikChange Site-Directed Mutagenesis Kit (Stratagene, Inc)., with a “doped” oligodeoxyribonucleotide primer, a complementary wild-type oligodeoxyribonucleotide primer, and pRL663 as template (primers at 75 nM; all other components at concentrations as specified by the manufacturer). Mutagenized plasmid DNA was introduced into cells, and plasmid-linked MccJ25^{r} clones were identified and characterized, as in the preceding section.

Complementation Assays:

Strain 397c [rpoC^{ts} 397 argG thi lac (λcI₈₅₇h₈₀Sₜ₆₈dlac+); Christie et al., 1996) was transformed with pRL663 or a pRL663 derivative, transformants (˜10⁴ cells) were applied to LB-agar plates (Sambrook and Russell (2001)) containing 1 μg/ml MccJ25 and 200 μg/ml ampicillin, plates were incubated 16 h at 37° C., and bacterial growth was scored.

Minimum Bacteriocidal Concentration (MBC) Assays:

Strain DH5α [hsdR17 recA1 relA1 endA1 gyrA96 gal deoR phoA supE44 thi Δ(lacZYA-argF)U169 ø80dlacZΔM15; Invitrogen, Inc.] was transformed with pRL663 or a pRL663 derivative, transformants (10⁶ cells) were incubated 2 h at 37° C. in 1 ml LB (Id.) containing 0.01, 0.05, 0.1, or 1 mg/ml MccJ25; aliquots (10 μl) were applied to LB-agar plates (Id.) containing 200 μg/ml ampicillin; plates were incubated 16 h at 37° C.; and colonies were counted. The MBC was defined as the lowest concentration of MccJ25 that yielded a colony count of <5.

### Example 2

### Probe-Labeled Derivatives of Peptide Antibiotic MccJ25 and Assays Therewith

One aspect of the invention provides probe-labelled derivatives of the peptide antibiotic MccJ25 (MccJ25)

The present invention also provides a composition comprising a compound according to the general structural formula (I): J-Z-X, wherein J is MccJ25 or a substituted and/or truncated derivative thereof, Z is a covalent linker or is absent, and X is a detectable group. One aspect of the present invention, provides a composition comprising a compound according to the general structural formula (I) wherein X is selected from the group consisting of a fluorescent moiety, a phosphorescent moiety, a luminescent moiety, an absorbent moiety, a photosensitizer, a spin label, a radioisotope, an isotope detectable by nuclear magnetic resonance, a paramagnetic atom, a heavy atom, a hapten, a crosslinking agent, a cleavage agent, and combinations thereof. Another aspect of the present invention, provides a composition comprising formula (I) wherein X is a fluorescent moiety. Another aspect of the present invention, provides a composition comprising formula (I) wherein X is a cyanine dye. Another aspect of the present invention, provides a composition comprising formula (I) wherein X is a Cy3. Another aspect of the present invention, provides a composition comprising formula (I) wherein X is a Cy5.

The present invention also provides a composition comprising a derivative of MccJ25 having a detectable group incorporated at position 5, according to the general structural formula (II): [O₅-Z-X]J, wherein O is an amino acid or amino acid derivative, Z is a covalent linker or is absent, and X is a detectable group. One aspect of the present invention, provides a composition according to formula) (II) wherein O is lysine; and wherein X is selected from the group consisting of a fluorescent moiety, a phosphorescent moiety, a luminescent moiety, an absorbent moiety, a photosensitizer, a spin label, a radioisotope, an isotope detectable by nuclear magnetic resonance, a paramagnetic atom, a heavy atom, a hapten, a crosslinking agent, a cleavage agent, and combinations thereof. A preferred aspect of the present invention, provides a composition according to formula (II) wherein X is a fluorescent moiety. An especially preferred aspect of the present invention, provides a composition according to formula (II) wherein X is a cyanine dye. An especially preferred aspect of the present invention provides a composition according to said formula (II) wherein X is Cy3 or Cy5.

The present invention also provides a composition comprising a derivative of MccJ25 having a detectable group incorporated at position 13, according to the general structural formula (III): [O₁₃-Z-X]J, wherein O is an amino acid or amino acid derivative, Z is a covalent linker or is absent, and X is a detectable group. One aspect of the present invention, provides a composition according to formula) (III) wherein O is lysine; and wherein X is selected from the group consisting of a fluorescent moiety, a phosphorescent moiety, a luminescent moiety, an absorbent moiety, a photosensitizer, a spin label, a radioisotope, an isotope detectable by nuclear magnetic resonance, a paramagnetic atom, a heavy atom, a hapten, a crosslinking agent, a cleavage agent, and combinations thereof. A preferred aspect of the present invention, provides a composition according to formula (III) wherein X is a fluorescent moiety. An especially preferred aspect of the present invention, provides a composition according to formula (III) wherein X is a cyanine dye. An especially preferred aspect of the present invention provides a composition according to said formula (III) wherein X is Cy3 or Cy5.

The present invention also provides a composition comprising a derivative of MccJ25 having a detectable group incorporated at position 15, according to the general structural formula (IV): [O₁₅-Z-X]J, wherein O is an amino acid or amino acid derivative, Z is a covalent linker or is absent, and X is a detectable group. One aspect of the present invention, provides a composition according to formula) (IV) wherein O is lysine; and wherein X is selected from the group consisting of a fluorescent moiety, a phosphorescent moiety, a luminescent moiety, an absorbent moiety, a photosensitizer, a spin label, a radioisotope, an isotope detectable by nuclear magnetic resonance, a paramagnetic atom, a heavy atom, a hapten, a crosslinking agent, a cleavage agent, and combinations thereof. A preferred aspect of the present invention provides a composition according to formula (IV) wherein X is a fluorescent moiety. An especially preferred aspect of the present invention provides a composition according to formula (IV) wherein X is a cyanine dye. An especially preferred aspect of the present invention provides a composition according to said formula (IV) wherein X is Cy3 or Cy5.

The present invention also provides a composition comprising a derivative of MccJ25 having a detectable group incorporated at position 17, according to the general structural formula (V): [O₁₇-Z-X]J, wherein O is an amino acid or amino acid derivative, Z is a covalent linker or is absent, and X is a detectable group. One aspect of the present invention, provides a composition according to formula) (V) wherein O is lysine; and wherein X is selected from the group consisting of a fluorescent moiety, a phosphorescent moiety, a luminescent moiety, an absorbent moiety, a photosensitizer, a spin label, a radioisotope, an isotope detectable by nuclear magnetic resonance, a paramagnetic atom, a heavy atom, a hapten, a crosslinking agent, a cleavage agent, and combinations thereof. A preferred aspect of the present invention, provides a composition according to formula (V) wherein X is a fluorescent moiety. An especially preferred aspect of the present invention provides a composition according to formula (V) wherein X is a cyanine dye. An especially preferred aspect of the present invention provides a composition according to said formula (V) wherein X is Cy3 or Cy5.

The present invention also provides a composition comprising a compound according to any one of the general structural formulas (I), (II), (III), (IV), or (V) for a) analysis, synthesis, screening, or design of ligands of RNAP; b) analysis, synthesis, screening, or design of modulators of RNAP activity; c) analysis, synthesis, screening, or design of modulators of bacterial gene expression; and d) analysis, synthesis, screening, or design of modulators of bacterial growth.

In a preferred embodiment, the invention provides for the use of a compound according to any one of the general structural formulas (I), (II), (III), (IV), or (V) in an assay assessing the ability of a molecule, or set of molecules, to displace said compound from a bacterial RNAP, or a fragment thereof, or to compete with said compound for binding to a bacterial RNAP, or a fragment thereof.

In an especially preferred embodiment, the invention provides for the use of a compound according to any one of the general structural formulas (I), (II), (III), (IV), or (V) in a homogeneous assay FRET assay measuring the ability of a molecule, or set of molecules, to displace said compound from a bacterial RNAP, or a fragment thereof, or to compete with said compound for binding to a bacterial RNAP, or a fragment thereof.

The present invention provides for the preparation of [Cy3-Lys5]MccJ25 and [Cy3-Lys13]MccJ25 (compounds according to structural formulas (II) and (III)) and demonstration that these compounds bind to and inhibit RNAP with high potency. The present invention also provides for the preparation of [Lys5]MccJ25, [Lys13]MccJ25, [Lys15]MccJ25, and [Lys17]MccJ25 (intermediates in synthesis of compounds according to structural formulas (I)-(V)) and demonstration that the compounds bind to and inhibit RNAP with high potency.

The present invention also provides for the use of at least one of [Cy3-Lys5]MccJ25 and [Cy3-Lys13]MccJ25 (compounds according to structural formulas (II) and (III)) in a homogeneous FRET assay measuring the ability of a molecule, or set of molecules, to displace said compound from a bacterial RNAP, or a fragment thereof, or to compete with said compound for binding to a bacterial RNAP, or a fragment thereof.

Another aspect of the invention provides assays employing probe-labeled derivatives of MccJ25.

An especially preferred aspect of the invention provides a FRET competition assay employing a fluorochrome-labeled derivative of MccJ25 and a fluorochrome-labeled derivative.

Another aspect of the invention provides use of assays employing probe-labeled derivatives of MccJ25 as a means to identify agents that bind within the RNAP secondary channel.

Another aspect of the invention provides use of assays employing probe-labeled derivatives of MccJ25 as a means to characterize agents that bind within the RNAP secondary channel.

MccJ25 Binds to RNAP:

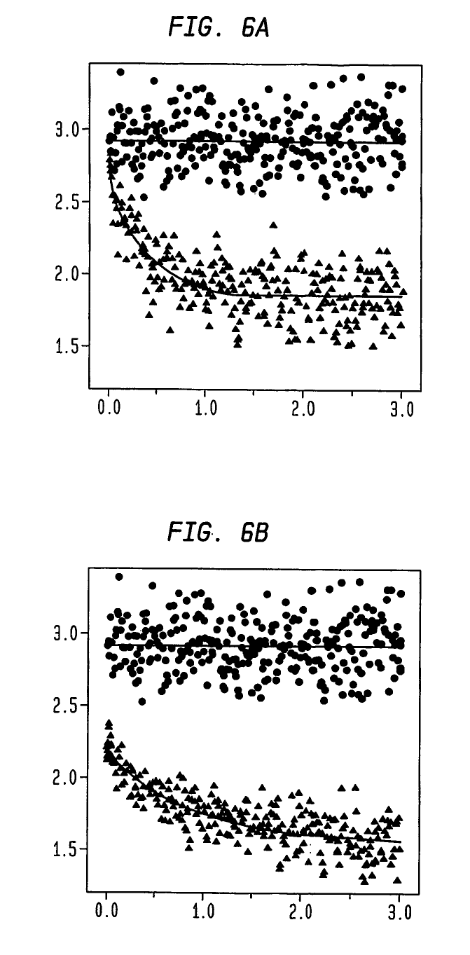

To determine whether MccJ25 binds to RNAP, fluorescence resonance energy transfer (FRET) binding experiments were performed. A fluorescent-probe-labelled MccJ25 derivative, [Cy3-Lys13]MccJ25 was prepared ands was verified to be functional in transcription inhibition. A fluorescent-probe-labeled RNAP derivative, DAC-σ⁷⁰ RNAP holoenzyme, was then titrated with [Cy3-Lys13]MccJ25 in the concentration range 0-20 μM, and FRET was monitored. The results reveal a large, saturable increase in FRET, indicating that [Cy3-Lys13]MccJ25 binds to RNAP (FIG. 5A, filled circles). Analysis of the concentration-dependence of the increase in FRET indicates that the equilibrium dissociation constant, K_{d}, for interaction of [Cy3-Lys13]MccJ25 with RNAP is 1.4 μM (FIG. 5A). Stopped-flow analysis of the kinetics of interaction of [Cy3-Lys13]MccJ25 with RNAP indicates that the association rate constant, kₒₙ, is 7×10⁵ M⁻¹ s⁻¹, and the dissociation rate constant, k_{off}, is 0.7 s⁻¹ (FIG. 6). The value of K_{d} estimated from stopped-flow analysis is 1 μM (K_{d}=k_{off}/kₒₙ), which, within experimental error, agrees with the value of K_{d} estimated from direct titration.

MccJ25 Binds to RNAP with K_{d} Comparable to Kᵢ:

To determine the equilibrium dissociation constant, K_{d}, for interaction of native, unlabeled MccJ25 with RNAP, FRET competition experiments were performed. Experiments were initiated with complexes of [Cy3-Lys13]MccJ25 and DAC-σ⁷⁰ RNAP holoenzyme, challenged complexes with unlabelled MccJ25 in the concentration range 0-20 μM, and monitored FRET. The results reveal a concentration-dependent decrease in FRET, indicating that unlabelled MccJ25 competes with [Cy3-Lys13]MccJ25 for binding to RNAP (FIG. 5B). Analysis of the concentration dependence of the decrease in FRET and application of the Cheng-Prusoff equation (Cheng and Prusoff, (1973) Biochem. Pharmacol. 22, 3099-3108), indicates that the equilibrium dissociation constant, K_{d}, for interaction of unlabeled MccJ25 with RNAP is 0.5 μM (FIG. 5B). Within experimental error, K_{d} for interaction of unlabeled MccJ25 with RNAP is equal to Kᵢ for inhibition of transcription (1.2±0.3 μM; FIG. 3E,F). It is concluded that the same binding process is monitored by the FRET experiments as is monitored by the transcription-inhibition experiments.

Binding Requires the Genetically Defined Determinant within the RNAP Secondary Channel:

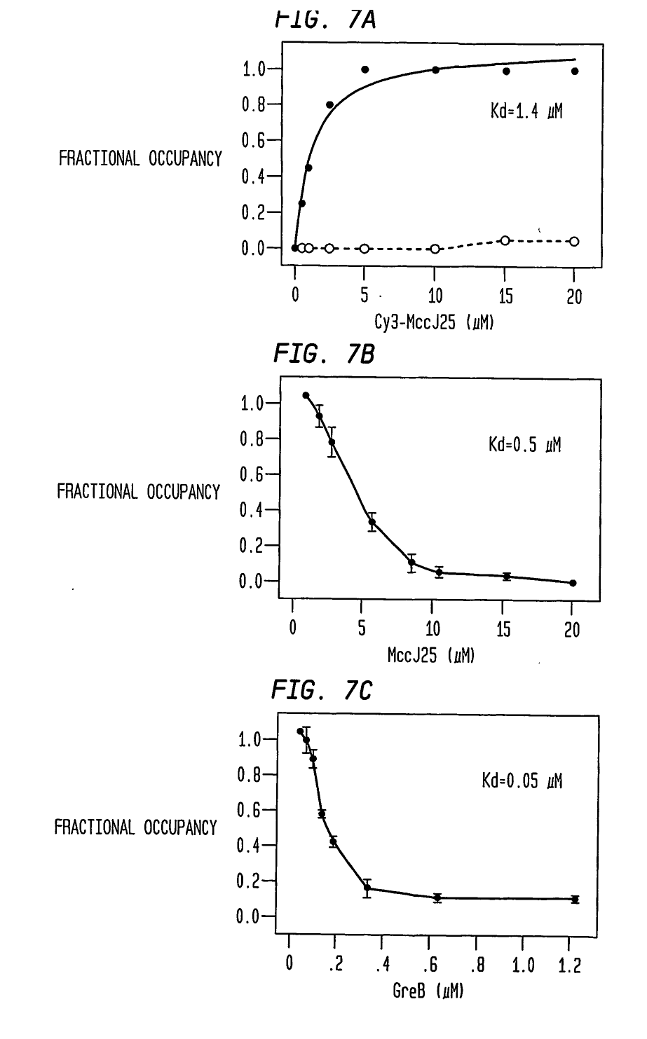

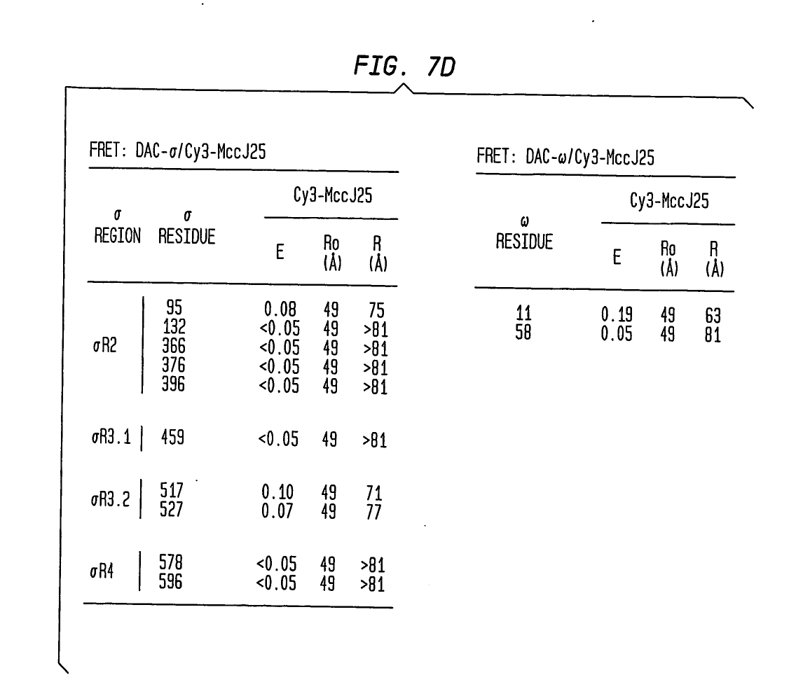

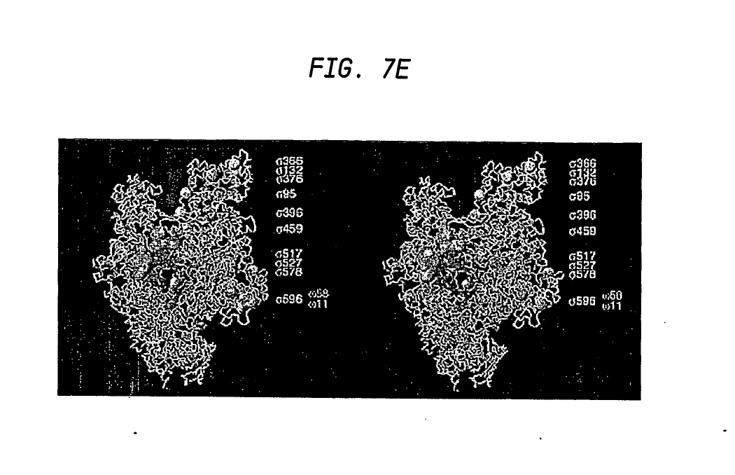

To determine whether binding of MccJ25 requires the genetically defined determinant within the RNAP secondary channel (FIG. 5B-E), FRET binding experiments were performed with [Cy3-Lys13]MccJ25 and a fluorescent-probe-labelled RNAP derivative having a single-amino-acid substitution within the determinant (Thr931→Ile; see FIG. 3C and Table 5; see also Delgado et al., (2001); Yuzenkova et al., (2002)). [Cy3-Lys13]MccJ25 exhibited no detectable interaction with the substituted RNAP derivative in the concentration range analyzed (FIG. 7A, open circles). It is concluded that binding of MccJ25 requires the genetically defined determinant within the RNAP secondary channel. It further is concluded that the genetically defined determinant represents a binding determinant for MccJ25 (as opposed to a conformational determinant required for MccJ25 function but not for MccJ25 binding).

Binding Occurs within the RNAP Secondary Channel: Competition with GreB:

To test the hypothesis that [Cy3-Lys13]MccJ25 binds within the RNAP secondary channel, FRET competition experiments were performed with [Cy3-Lys13]MccJ25 and the transcript-cleavage factor GreB, which has been shown to bind within the RNAP secondary channel (Opalka et al., (2003) Cell 114, 335-345; Laptenko et al., (2003) EMBO J. 22, 6322-6334; Sosunova et al., (2003) Proc Natl Acad Sci USA 100, 15469-15474). Experiments were initiated with complexes of [Cy3-Lys13]MccJ25 and DAC-σ⁷⁰ RNAP holoenzyme, complexes were challenged with GreB in the concentration range 0-1 μM, and FRET was monitored. The results show that GreB competes with [Cy3-Lys13]MccJ25 for binding to RNAP (FIG. 7C). The calculated value of K_{d} for interaction of GreB with RNAP is 0.05 mM, which, within experimental error, agrees with previously reported values (Koulich et al., (1997) J. Biol. Chem. 272, 7201-7210) (FIG. 7C). It is concluded that binding of [Cy3-Lys13]MccJ25 to RNAP and binding of GreB to RNAP are mutually exclusive. It further is concluded that the binding location for MccJ25 overlaps the binding location for GreB: i.e., that MccJ25 binds within, or immediately adjacent to, the RNAP secondary channel.

Binding Occurs within the RNAP Secondary Channel: Systematic FRET:

To test further the hypothesis that [Cy3-Lys13]MccJ25 binds within the RNAP secondary channel, and to define the location of [Cy3-Lys13]MccJ25 relative to RNAP within the [Cy3-Lys13]MccJ25-RNAP complex by a direct, physical approach, systematic FRET measurements of distances and distance-restrained docking were performed (methods essentially as in Meckler et al., (2002) Cell 108, 599-614). FRET was used to measure the distances between the Cy3 probe of [Cy3-Lys13]MccJ25 and DAC probes incorporated into RNAP holoenzyme at each of ten sites in σ⁷⁰ (FIG. 7D, left) and each of two sites in ω (FIG. 7D, right). Automated distance-restrained docking was used to define locations of the Cy3 probe of [Cy3-Lys13]MccJ25 relative to RNAP holoenzyme consistent with the measured distances (dark spheres in FIG. 7E). The results indicate that the Cy3 probe of [Cy3-Lys13]MccJ25 is located in the RNAP secondary channel, at the external mouth of the RNAP secondary channel (the mouth distal to the RNAP active center; dark spheres in FIG. 7E). The Cy3 probe of [Cy3-Lys13]MccJ25 is located immediately adjacent to the genetically defined determinant within the RNAP secondary channel (dark spheres in grape-like clusters and dark van der Waals surfaces in FIG. 7E). MccJ25 binds within, or immediately adjacent to, the RNAP secondary channel.

MccJ25 Obstructs the RNAP Secondary Channel:

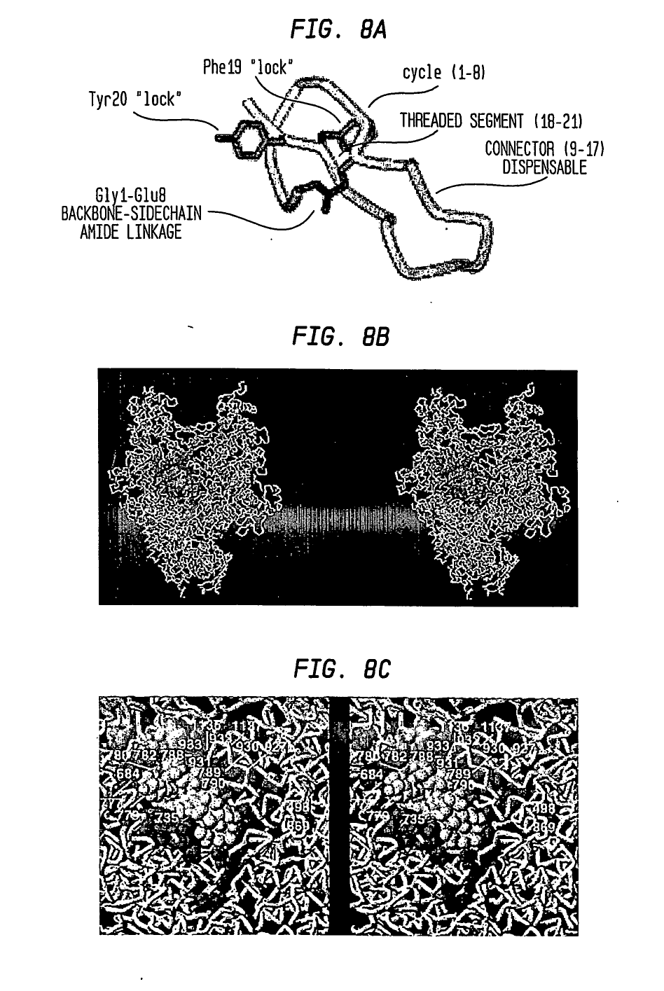

MccJ25 is a 21-residue “lariat protoknot,” consisting of an 8-residue cyclic segment followed by a 13-residue linear segment that loops back and threads through the cyclic segment (FIG. 8A; Bayro et al., (2003); Rosengren et al., (2003); Wilson et al., (2003)). MccJ25 has dimensions of ˜27 Å×˜14 Å and is roughly comma-shaped, with the cycle and threaded segment (residues 1-8 and 18-21) corresponding to the head of the comma, and with the connector segment (residues 9-17) corresponding to the tail of the comma (FIG. 8A).

MccJ25 exhibits structural complementarity, in both size and shape, to the RNAP secondary channel. In cross-section, the RNAP secondary channel has dimensions of ˜28 Å×˜14 Å and is roughly comma-shaped, with the secondary-channel sub-region bounded by the β′ D/E helix, E helix, F-helix, and G-helix and loop corresponding to the head of the comma, and with the secondary-channel sub-region bounded by β′ residues 498-504, 732-733, 922-926, and 1244-1248 corresponding to the tail of the comma.

This structural complementarity was used to dock the structure of MccJ25 into the structure of RNAP in order to construct a model for the structure of the MccJ25-RNAP complex (FIG. 8B,C). In the resulting model, the cycle and threaded segment of MccJ25 (the head of the comma) is placed in the secondary-channel sub-region bounded by the β′ D/E helix, E helix, F-helix, and G-helix and G-loop; and the connector segment of MccJ25 (the tail of the comma) is placed in the secondary-channel sub-region bounded by β′ residues 498-504, 732-733, 922-926, and 1244-1248 (FIG. 8B,C). The resulting model is supported by four observations. First, the model is sterically acceptable (FIG. 8B,C). Second, the model accounts for genetic data defining critical determinants of RNAP (FIG. 5; Tables 2, 5)—placing MccJ25 in contact with, or adjacent to, the majority of sites of substitutions conferring MccJ25-resistance (FIG. 8B,C), and, in particular, placing the cycle and threaded segment of MccJ25 in contact with, or adjacent to, the majority of sites of substitutions conferring highest-level MccJ25-resistance (labelled residues in FIG. 8C). Third, the model accounts for genetic and biochemical data defining critical determinants of MccJ25 (unpublished data)—placing essential residues of MccJ25 on the face of MccJ25 directed toward RNAP, and placing non-essential residues of MccJ25 on the face of MccJ25 directed toward solvent. Fourth, the model accounts for systematic-FRET/distance-restrained-docking data defining possible locations of the Cy3 probe of Cy3-MccJ25 (FIG. 3E)—placing the attachment site of Cy3 on the face of MccJ25 directed toward the external mouth of the secondary channel, at a position of MccJ25 close to the external mouth of the secondary channel.

The most striking feature of the model is that it suggests that MccJ25 essentially completely seals the RNAP secondary channel—like a “cork in a bottle” (FIG. 8B,C). The model suggests that binding of MccJ25 would block passage of molecules the size of NTPs and possibly also would block passage of smaller molecules. It has been proposed that NTPs pass through the RNAP secondary channel to enter the RNAP active center (Zhang et al., (1999); Cramer et al., (2000); Korzheva et al., (2000); Gnatt et al., (2001); see however, Nedialkov et al., (2003) J. Biol. Chem. 278, 18303-18312), and that abortive RNA fragments, edited RNA fragments, and backtracked RNA segments pass through the secondary channel to exit the active center (Cramer et al., (2000); Korzheva et al., (2000); Gnatt et al., (2001)). The model suggests that binding of MccJ25 would block these transactions. It also has been proposed that pyrophosphate may pass through the RNAP secondary channel to exit the RNAP active center. The model suggests that binding of MccJ25 might block this transaction. Finally, it has been shown that the transcript-cleavage factors GreA and GreB must enter the secondary channel to access the active center (Opalka et al., (2003); Laptenko et al., (2003); Sosunova et al., (2003)). The model suggests that binding of MccJ25 would block function of transcript-cleavage factors, consistent with the observed binding competition between Cy3-MccJ25 and GreB (FIG. 7C).

Experimental Procedures:

Plasmids:

Plasmids pTUC202-K5, pTUC202-K13, pTUC202-K15, and pTUC202-K7, which carry genes for synthesis and export of [Lys5]MccJ25, [Lys13]MccJ25, [Lys15]MccJ25, and [Lys17]MccJ25, were constructed from pTUC202 (Solbiati et al., (1996)) by use of site-directed mutagenesis (QuikChange Site-Directed Mutagenesis Kit; Stratagene, Inc.). Plasmid pREII-NHα encodes N-terminally hexahistidine-tagged E. coli RNAP α subunit under control of tandem lpp and ′lacUV5 promoters (Niu et al., (1996)). Plasmids pET21a-β′ encoding β′ for use in reconstitution of RNAP was constructed by replacement of the NdeI-HindIII segment of pET21a (Novagen, Inc.) by the NdeI-HindIII rpoC segment of pET15b-β′ (K. Severinov, personal communication). Plasmids pMKSe2, pHTT7f1-NHα, pT7ω, and pHTT7f1-σ, encoding β, N-terminally hexahistidine-tagged α, ω, and σ⁷⁰ for use in reconstitution of RNAP, were described in Tang et al. (1995) Proc. Natl. Acad. Sci. USA 92, 4902-4906, Martin et al. (1992) J Biol Chem 67, 20175-20180, Naryshkin et al., (2001) Mol. Biol. 148, 337-361, and Mekler et al., (2002)). Plasmids encoding [Ile931]β′and ω derivatives with single Cys residues at positions 11 and 58 for use in reconstitution of RNAP were constructed from pET21a-β′ and pT7ω by use of site-directed mutagenesis. Plasmid pGEMD(-Cys), encoding a σ⁷⁰ derivative with no Cys residues, and plasmids encoding σ⁷⁰ derivatives with single Cys residues at positions 132, 366, 376, 396, 459, 517, 527, and 596, were described in Owens et al., (1998) Proc. Natl. Acad. Sci. 95, 6021-6026, Callaci et al., (1998) J. Biol. Chem. 273, 32995-33001, Bown et al., (1999) J. Biol. Chem. 274, 2263-2270, and Mukhopadhyay et al., 2001. A plasmid encoding a σ⁷⁰ derivative with a single Cys residue at position 95 was constructed from pGEMD(-Cys) by use of site-directed mutagenesis.

MccJ25:

MccJ25 was prepared as in Example 1.

Cy3-MccJ25:

[Cy3-Lys5]MccJ25 and [Cy3-Lys13]MccJ25 were prepared using Lys-specific chemical modification. Reaction mixtures contained (200 μl): 1 mM (Lys5]MccJ25 or [Lys13]MccJ25 derivative (prepared as for MccJ25 in Example 1, but using plasmid pTUC202-5K or plasmid pTUC202-13K), 2 mM Cy3 NHS-ester (Amersham-Pharmacia Biotech), and 0.5% (v/v) triethylamine in dimethylsulfoxide. Following 15 h at 4° C., products were purified by reversed-phase HPLC on a C18, 5 μm, 300 Å column (Rainin), with solvent A=0.1% trifluoroacetic acid, solvent B=90% acetonitrile and 0.1% trifluoroacetic acid, and gradient=25-40% solvent B in solvent A in 30 min, and flow rate=1 ml/min. Fractions containing [Cy3-Lys5]MccJ25 [Cy3-Lys13]MccJ25 (retention time ˜29 min; detected by UV absorbance at 276 nm) were pooled, lyophilized, re-dissolved in 500 μl 30% methanol, and stored in aliquots at −20° C.

RNAP:

RNAP core and holoenzyme were prepared as in Niu et al., (1996)). [Ile931]β′ RNAP core and holoenzyme were prepared from strain 397c [rpoC^{ts}397 argG thi lac (λcI₈₅₇h₈₀Sₜ₆₈dlac⁺); Christie et al., (1996))] transformed with a pRL663 derivative encoding [Ile931] β′-RNAP using analogous procedures. Yields typically were 6 mg. Purities typically were >95%.

DAC-σ⁷⁰ RNAP:

DAC-σ⁷⁰ was prepared using Cys-specific chemical modification. Reaction mixtures contained (1 ml): 20 μM single-Cys σ⁷⁰ derivative [prepared as in Mukhopadhyay et al., (2001); subjected to solid-phase reduction on Reduce-Imm (Pierce) per manufacturer's instructions immediately before use], 200 μM N-(7-dimethylamino-4-methylcoumarin-3-yl)maleimide (Molecular Probes), 100 mM sodium phosphate (pH 8.0), and 1 mM EDTA. Following 1 h at 4° C., products were purified by gel-filtration chromatography on Bio-Gel P6DG (Bio-Rad), and stored in 20 mM Tris-HCl (pH 7.9), 100 mM NaCl, 0.1 mM. EDTA, 0.1 mM DTT, and 50% glycerol at −20° C. Efficiencies of labelling, determined by measurement of UV-absorbance, were ˜90%; site-specificities of labelling, determined by comparison to products of control reactions with wild-type σ⁷⁰, were ˜90%.

DAC-σ⁷⁰ RNAP holoenzyme and DAC-σ⁷⁰ [Ile931]β′ RNAP holoenzyme were prepared by incubation of 5 pmol unlabelled RNAP core and [Ile931]β′ RNAP core, respectively, with 4 pmol DAC-σ⁷⁰ in 20 μl TB for 20 min at 25° C.

DAC-ω RNAP:

DAC-ω was prepared using Cys-specific chemical modification. Reaction mixtures contained (1 ml): 20 μM single-Cys ω derivative [prepared essentially as described for ω in Mekler et al., (2002); subjected to solid-phase reduction on Reduce-Imm (Pierce) per manufacturer's instructions immediately before use], 200 μM N-(7-dimethylamino-4-methylcoumarin-3-yl)maleimide (Molecular Probes), 100 mM sodium phosphate (pH 8.0), 1 mM EDTA, and 6 M guanidine hydrochloride. Following 1 h at 25° C., products were purified by gel-filtration chromatography on Bio-Gel P6DG (Bio-Rad,). Site-specificities of labelling, determined by comparison to products of control reactions with wild-type ω, were ˜90%.

DAC-ω RNAP holoenzyme and DAC-ω [Ile931]β′ RNAP were prepared by reconstitution as described for unlabelled RNAP holoenzyme in Naryshkin et al., 2001, using 500 μg β′ or [Ile931]β′, 300 μg β, 30 μg hexahistidine-tagged α, and 250 μg σ⁷⁰, including 100 μg DAC-ωₙ), and including a final incubation for 45 min at 30° C. Products were purified using metal-ion-affinity chromatography on Ni:NTA-agarose and ion-exchange chromatography on Mono-Q (methods essentially as in Naryshkin et al., 2001 and Niu et al., (1996); concentrated (methods essentially as in Naryshkin et al., (2001)); and stored in 20 mM Tris-HCl (pH) 7.9, 100 mM NaCl, 0.1 mM EDTA, 0.1 mM DTT, and 50% glycerol at −20° C.

FRET Binding Assays:

Assay mixtures (50 μl) contained 100 nM DAC-σ⁷⁰ RNAP holoenzyme (labeled at residue 517 of σ⁷⁰) and 0-20 μM Cy3-MccJ25 in TB at 25° C. Fluorescence emission intensities were measured before and 5 min after addition of 1 μl 2 mM unlabeled MccJ25 [excitation wavelength=385 nm; emission wavelength=465 nm; excitation and emission slit widths=5 nm; QuantaMaster QM1 spectrofluorometer (PTI)]. Efficiencies of fluorescence resonance energy transfer (E) and fractional saturations (e) were calculated as:
E=1−F/Fₓ (2)
θ=E/Eₘₐₓ (3)
where F and Fₓ are emission intensities before and after addition of 40 μM unlabeled MccJ25, and where Eₘₐₓ is E at saturation. The equilibrium dissociation constant (K_{d,Cy3-MccJ}25) was extracted by non-linear regression using the equation:
θ=[Cy3-MccJ25]/(K_{d,Cy3-MccJ25}+[Cy3-MccJ25]) (4)
where [Cy3-MccJ25] is the concentration of Cy3-MccJ25.

To assess kinetics of binding, using a stopped-flow instrument (SFM-4/QS; Bio-Logic), 100 μl, 5 μM or 10 μM Cy3-MccJ25 in TB was mixed with 100 μl 100 nM DAC-σ⁷⁰ RNAP in TB, and fluorescence emission intensity was monitored as a function of time following mixing [excitation wavelength=400 nm; emission wavelength=455 nm; QuantaMaster QM1 spectrofluorometer (PTI); data from four shots averaged]. The observed rate of complex formation, k_{obs}, the association rate constant, kₒₙ, and the dissociation rate constant, k_{off}, were calculated as:
F=Fₒ+ae^{-k}^{_{obs}}^{t} (5)
kₒₙ=(k_{obs,1}−k_{obs,2})/([Cy3-MccJ25]₁−[Cy3-MccJ25]₂) (6)
k_{off}=kₒₙ[Cy3-MccJ25]₁−k_{obs,1} (7)
where F is fluorescence emission intensity at time, t; Fₒ+a is the fluorescence emission intensity at t=0; Fₒ is fluorescence emission intensity at t=∞; and k_{obs,1} and k_{obs,2} are observed rates at [Cy3-MccJ25]₁ and [Cy3-MccJ25]₂
Fret Competition Assays:

Assay mixtures (50 μl) contained 100 nM DAC-σ⁷⁰ RNAP holoenzyme (labelled at residue 517 of σ⁷⁰), 5 μM Cy3-MccJ25, and 0-20 μM competitor [MccJ25 or GreB (prepared as in Koulich et al., (1997)] in TB at 25° C. Fluorescence emission intensities were measured before and 5 min after addition of 1 μl 2 mM unlabeled MccJ25, and E and θ were calculated as above. Equilibrium dissociation constants (K_{d}) were calculated as:
K_{d}=IC₅₀/(1+[Cy3-MccJ25]/K_{d,Cy3-MccJ25}) (8)
where IC₅₀ is the concentration of competitor that yields half-maximal competition.
Fret Distance Measurements:

Assay mixtures (50 μl) contained 100 nM DAC-labelled RNAP holoenzyme derivative or corresponding DAC-labelled [Ile931]β′ RNAP holoenzyme derivative in 50 mM Tris-HCl (pH 8.0), 800 mM KCl, 10 mM MgCl₂, 1 mM dithiothreitol, 10 μg/ml bovine serum albumin, and 5% glycerol at 25° C. Fluorescence emission intensities were measured before and 5 min after addition of 2 μl 250 μM Cy3-MccJ25, and values of E were calculated as:
E=(1−F_{R}/F_{o,R})−(1−F_{R′}/F_{o,R′}) (9)
where F_{o,R} and F_{R} are emission intensities before and after addition of Cy3-MccJ25 to DAC-labelled RNAP holoenzyme derivative, and F_{o,R′} and F_{R′} are emission intensities before and after addition of Cy3-MccJ25 to the corresponding DAC-labelled [Ile931]β′ RNAP holoenzyme derivative.
Donor-acceptor distances (R) were calculated as:
R=Rₒ[(1/E)−1]^{1/6} (10)
where Rₒ is the Förster parameter (49.4 Å in this study, calculated essentially as in Mekler et al., 2002).
Distance-Restrained Docking:

Distance-restrained docking was performed essentially as in Mekler et al., (2002).

DAC chromophores and linkers of DAC-labelled RNAP holoenzyme derivatives were modelled into the structure of Thermus thermophilus RNAP holoenzyme Vassylyev et al., (2002) Nature 417 712-719 (PDB accession 1IW7) using IMPACT (Schröder, Inc.). For each DAC chromophore and linker, sterically-allowed conformations were identified [sampling all linker torsion angles in 30° increments and accepting all conformations with van der Waals energy<5000 kcal/mol in the OPLS all-atom force field (Jorgenson et al., (1996) J. Am. Chem. Soc. 118, 11225-[1236]; and, for each sterically-allowed conformation, a pseudoatom corresponding to the center of the DAC chromophore was defined.

Markov-chain Monte Carlo simulations (Metropolis et al., (1953) J. Chem. Phys. 21, 1087-1091; Hastings, (1970) Biometrika 57, 97-109) employing FRET-derived distance restraints were performed to define allowed positions of a pseudoatom representing the center of the Cy3 chromophore of Cy3-MccJ25 (acceptor pseudoatom, a) relative to the positions of pseudoatoms representing the centers of DAC chromophores of DAC-labelled RNAP holoenzyme derivatives (donor pseudoatoms, d).

For each configuration Y, for each donor-acceptor pair i, the distances between the acceptor pseudoatom, a, and each donor pseudoatom, dm, were assessed, and were used to calculate a configuration-specific, donor-acceptor-pair-specific mean FRET efficiency (E_{Y,i}^{calc}) and mean distance (R_{Y,i}^{calc}):

$$\begin{matrix}{E}_{Y , i}^{calc} = \frac{1}{M} {\sum }_{m = 1}^{M} {[ 1 + {( \frac{{R}_{{d}_{m} a}}{{R}_{o , i}} )}^{6} ]}^{- 1} & ( 1 ) \\ {R}_{Y , i}^{calc} = {{R}_{o , i} ( \frac{1}{{E}_{Y , i}^{calc}} - 1 )}^{1 / 6} & ( 2 )\end{matrix}$$

where M is the total number of donor pseudoatoms aₘ (˜300 in this work), R is the donor-acceptor distance, and R_{o,i} is the Förster parameter for the donor-acceptor pair. π_{FRET}(Y,i), the configuration-specific, donor-acceptor-pair-specific penalty function for deviation between the calculated mean distance (R_{Y,i}^{calc}) and the experimental distance (Rᵢ^{obs}), and π_{FRET} (Y), the corresponding configuration-specific, global penalty function, were calculated as:

$${π}_{FRET} ( Y , i ) = {( 2 π {σ}_{i}^{2} )}^{- 1 / 2} exp [ \frac{- ( {R}_{Y , i}^{calc} - {R}_{i}^{obs} )}{2 {σ}_{i}^{2}} ] \qquad ( 3 )$$

where σᵢ is the error associated with Rᵢ^{obs} (10% of Rᵢ^{obs} in this work). Values of Rᵢ^{obs}>81 Å (Eᵢ^{obs}<0.05) were treated as bounds, such that:

$${π}_{FRET} ( Y , i ) = \{ \begin{matrix}{( 130 π )}^{- 1 / 2} exp [ \frac{- ( {R}_{Y , i}^{calc} - 81 Å )}{2 {σ}_{i}^{2}} ] , & if {R}_{i}^{calc} \leq 81 Å \\ {( 130 π )}^{- 1 / 2} , & if {R}_{i}^{calc} > 81 Å\end{matrix} \qquad ( 4 )$$

π_{FRET}(Y), the corresponding configuration-specific, global penalty function, was calculated as:

$${π}_{FRET} ( Y ) = {\prod }_{i = 1}^{I} {π}_{FRET} ( Y , i ) \qquad ( 5 )$$

where I is the total number of donor-acceptor pairs (12 in this work).

Trial configurations (Y) were generated from previously accepted configurations (X) by translating the acceptor pseudoatom, a, along x-, y- and z-axes in a laboratory reference frame and were accepted with probability α(X,Y), as follows:

$$α ( X , Y ) = min [ 1 , \frac{{π}_{FRET} ( Y )}{{π}_{FRET} ( X )} ] \qquad ( 6 )$$

where π_{FRET}(X) and π_{FRET}(Y) are configuration-specific penalty terms for FRET violations, and where the translational sampling parameter was optimized to achieve acceptance rates between 35-65%. Three simulations with different initial conditions were performed. In each simulation, 10,000 trial configurations were generated.

Results were post-processed to eliminate configurations that place the acceptor pseudoatom less than 3 Å or more than 20 Å from the nearest RNAP Cα atom, and to rank the 40% of configurations with lowest π_{FRET}(Y) by a configuration-specific reduced Lennard-Jones potential (Levitt, (1976), E_{steric}(Y):

$${E}_{steric} ( Y ) = {\sum }_{k = J}^{K} ɛ [ 3 {( \frac{{r}_{o}}{{r}_{ak}} )}^{8} - 4 {( \frac{{r}_{o}}{{r}_{ak}} )}^{6} ] \qquad ( 7 )$$

where rⱼₖ is the configuration-specific distance between the acceptor pseudoatom and RNAP holoenzyme Cα atom k, K is the total number of RNAP holoenzyme Cα atoms, rₒ is the equilibrium inter-residue distance (10 Å), and ε is the well depth (0.15 kcal/mol).

### Example 3

### Minimized Derivatives of Peptide Antibiotic MccJ25

The present invention provides active “minimized” derivatives of the peptide antibiotic MccJ25. Such minimized MccJ25 derivatives have reductions in molecular mass of up to ˜50% (thereby increasing potency per mass by up to ˜200% and potentially improving solubility, bioavailablity, and target cell uptake) and reductions in number of atoms by up to ˜50% (thereby simplifying identification of the active pharmacore, potentially yielding narrower resistance spectra, and potentially yielding broader species spectra). As such, said minimized MccJ25 derivatives have uses as ligands of RNAP; modulators of RNAP; modulators of bacterial gene expression; and modulators of bacterial growth. These minimized MccJ25 derivatives also have utility in the analysis, synthesis, screening, and design of other ligands of RNAP, modulators of RNAP, modulators of bacterial gene expression, and modulators of bacterial growth.

Accordingly, one aspect of the invention provides a minimized derivative of MccJ25 that: (a) contains a discontinuity in the peptide backbone between residues 8 and 19; and (b) lacks one to about ten residues from the set consisting of residues 9-18 (termed MccJ25_{gap8/19})

In particular, the invention provides MccJ25_{gap8/19} derivatives lacking residues 11-12, 11-13, 11-14, 11-15, 11-16, and 11-17.

The present invention also provides a method of preparation of a minimized MccJ25 derivative involving proteolytic digestion of MccJ25. Such proteolytic digestion can be performed using an endopeptidase (preferably thermolysin) optionally in conjunction with at least one of an aminopeptodase, a dipeptidyl aminopeptidase, and another endopeptidase (preferably pronase).

A minimized MccJ25 derivative can be labeled with a detectable group as described above in Example 2 and used in a homogeneous FRET assay to determine the ability of a molecule, or set of molecules, to displace a labeled minimized MccJ25 derivative from a bacterial RNAP, or a fragment thereof, or to compete with a labeled minimized MccJ25 derivative for binding to a bacterial RNAP, or a fragment thereof.

MccJ25_{gap8/19} derivatives lacking residues 11-12, 11-13, 11-14, 11-15, 11-16, and 11-17 have been prepared (using proteolytic digestion of MccJ25 followed by reversed-phase HPLC). Binding assays (using FRET competition assays as in Example 2) and transcription assays (using fluorescence-detected abortive initiation assays as in Example 1) indicate that said derivatives bind to bacterial RNAP and inhibit bacterial transcription and with potencies indistinguishable from that of MccJ25. Bacteriocidal assays with the MccJ25_{gap8/19} derivative lacking residues 11-17 (using MBC assays as in Example 1) indicate that said derivative exhibits bacteriocidal activity indistinguishable from that of MccJ25. Analysis of resistance spectra with the MccJ25gape/19 derivative lacking residues 11-17 (using the full set of MccJ25-resistant mutants in Example 1) indicates that said derivative exhibits a narrower resistance spectrum than MccJ25—i.e., exhibits full, or nearly full, bacteriocidal activity for at least one MccJ25-resistant mutant.

### INDUSTRIAL APPLICABILITY

The invention has broad applications in analysis of RNAP structure and function, control of bacterial gene expression, control of bacterial growth, antibacterial chemistry, antibacterial therapy, and drug discovery.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

1. An analog of bacteriocidal peptide microcin J25 (MccJ25) that binds bacterial RNAP and inhibits bacterial RNAP activity and that: (a) contains a discontinuity in the peptide backbone between residues 8 and 19; and (b) lacks one to about ten residues from the set consisting of residues 9-18.

2. An analog according to claim 1 that: (a) contains a discontinuity in the peptide backbone between residues 10 and 18; and (b) lacks one to about seven residues from the set consisting of residues 11-17.

3. An analog according to claim 2 that lacks one of residues 11-12, residues 11-13, residues 11-14, residues 11-15, residues 11-16, and residues 11-17.

4. An analog according to claim 1 also containing a detectable group.

5. An analog according to claim 4 wherein the detectable group contains a chromophore.

6. An analog according to claim 4 wherein the detectable group contains a fluorophore.

7. An analog according to claim 4 wherein the detectable groups is Cy3.

8. A method for identifying an agent that binds to a bacterial RNAP homologous secondary channel amino acid sequence, comprising (a) preparing a reaction solution comprising the agent to be tested, a reference compound according to claim 1, and a first entity containing a bacterial RNAP homologous secondary channel amino acid sequence, and (b) detecting at least one of the presence, extent, concentration-dependence, or kinetics of competition by the agent for binding of the reference compound to the homologous secondary channel amino acid sequence.

9. The method of claim 8 wherein the first entity is an intact bacterial RNAP.

10. The method of claim 8 wherein the first entity is a fragment of a bacterial RNAP.

11. The method of claim 8 wherein the first entity is a derivative of Escherichia coli RNAP.

12. The method of claim 8 the first entity is a derivative of Bacillus subtilis RNAP.

13. The method of claim 8 wherein the reference compound contains a detectable group.

14. The method of claim 13 wherein the detectable group contains a chromophore.

15. The method of claim 13 wherein the detectable group contains a fluorophore.

16. The method of claim 8 further comprising measurement of FRET.

17. The method of claim 8 further comprising comparison of: (a) at least one of the presence, extent, concentration-dependence, or kinetics of the binding of the agent to the first entity; and (b) at least one of the presence, extent, concentration-dependence, or kinetics of the binding of the agent to a second entity that contains a derivative of a bacterial RNAP homologous secondary channel amino acid sequence having at least one substitution, insertion, or deletion.

18. The method of claim 17 wherein the second entity is a derivative of an intact bacterial RNAP.

19. The method of claim 17 wherein the second entity is a derivative of a fragment of a bacterial RNAP.

20. The method of claim 17 wherein the second entity is a derivative of Escherichia coli RNAP.

21. The method of claim 17 wherein the second entity is a derivative of Bacillus subtilis RNAP.

22. The method of claim 8 further comprising comparison of: (a) at least one of the presence, extent, concentration-dependence, or kinetics of binding of the agent to the first entity, and (b) at least one of the presence, extent, concentration-dependence, or kinetics of binding of the agent to a eukaryotic RNAP derivative.

23. The method of claim 17 wherein the eukaryotic RNAP derivative is a human RNAP derivative.

24. The method of claim 17 wherein the eukaryotic RNAP derivative is a human RNAP II derivative.

25. The method of claim 8 further comprising comparison of: (a) at least one of the presence, extent, concentration-dependence, or kinetics of binding of the agent to the first entity; and (b) at least one of the presence, extent, concentration-dependence, or kinetics of binding of MccJ25 to the first entity.
